(19) United States
(12) Patent Application Publication
(10) Pub. No.: US 2025/0064773 A1
(43) Pub. Date: Feb. 27, 2025

(54) TARGETING ERO1 ALPHA WITH AURONE DERIVATIVES

(71) Applicants: West Virginia University Board of Governors on behalf of West Virginia University, Morgantown, WV (US); Modulation Therapeutics, Inc., Tampa, FL (US)

(72) Inventors: Lori Hazelhurst, Morgantown, VA (US); Werner Geldenhuys, Morgantown, VA (US); Sridhar Reddy Kaulagari, Morgantown, WV (US)

(73) Assignees: West Virginia University Board of Governors on behalf of West Virginia University, Morgantown, WV (US); Modulation Therapeutics, Inc., Tampa, FL (US)

(21) Appl. No.: 18/720,324

(22) PCT Filed: Dec. 13, 2022
(86) PCT No.: PCT/US2022/081437

§ 371 Date: Jun. 14, 2024

Related U.S. Application Data
(60) Provisional application No. 63/265,374, filed on Dec. 14, 2021.
(60) Provisional application No. 63/267,751, filed on Feb. 9, 2022.

(51) Int. Cl. A61K 31/343; A61K 31/443

(52) U.S. Cl. CPC A61K 31/343; A61K 31/443

(57) ABSTRACT

Compounds and pharmaceutical compositions comprising a derivative of sulfuretin are provided that are inhibitors of the endoplasmic reticulum oxidoreductin-1 alpha. A method of treating a patient having cancer comprising administering to the patient a therapeutically effective amount of a compound that is a derivative of sulfuretin that are inhibitors of the endoplasmic reticulum oxidoreductin-1 alpha to a patient is disclosed.

### CROSS-REFERENCE TO RELATED APPLICATION

This utility non-provisional patent application claims the benefit of priority to U.S. Provisional Patent Application Ser. No. 63/265,374, filed Dec. 14, 2021, and to U.S. Provisional Patent Application Ser. No. 63/267,751, filed on Feb. 9, 2022. The entire contents of U.S. Patent Application Ser. Nos. 63/265,374 and 63/267,751 are incorporated by reference into this utility non-provisional patent application as if fully written herein.

### STATEMENT REGARDING FEDERALLY SPONSORED RESEARCH OR DEVELOPMENT

This invention was made with government support under Grant Nos. R44CA221554 and R01CA195727 awarded by the National Institutes of Health. The government has certain rights in the invention.

### BACKGROUND OF THE INVENTION

### 1. Field of the Invention

This invention provides compounds and pharmaceutical compositions that are derivatives of sulfuretin for use as inhibitors for the endoplasmic reticulum oxidoreductin-1alpha protein. This invention provides for a method of targeting Ero1α for treatment of cancer. The cancer may be lung cancer. The lung cancer may be non-small cell lung cancer. Ero1-α is a marker of poor clinical prognosis across multiple tumor types. Genetic depletion of Ero1α inhibits tumor growth.

### 2. Background Art

Lung cancer remains the most common cancer death worldwide at approximately 1.6 million deaths occurring each year. Non-small lung cancer treatment regimens have had increasing clinical benefit over the last two decades with the discovery of molecular drivers and development of tyrosine kinase inhibitors and immunotherapies¹. However, emergence of resistance to targeted and immunotherapies remains a clinical obstacle for increasing overall survival and improving patient outcomes²⁻⁵. Recently, increased expression of endoplasmic reticulum oxidoreductin-1 alpha (ERO1α) has been identified as a key player in promoting metastatic burden, proliferation, immune escape, and increased expression of ERO1α correlates with worse clinical outcomes in multiple tumor indications⁶⁻¹¹. ERO1α belongs to the flavoenzyme family of enzymes. Flavoenzymes are enzymes that meet two criteria; 1.) the enzyme must be an oxidoreductase and 2.) must contain flavin adenine dinucleotide (FAD) as a prosthetic group¹². Examples of flavoenzymes include ERO1α, ERO1β, LSD-1, MAO-A, and MAO-B. The endoplasmic reticulum (ER) resident enzyme ERO1α is known to form de novo disulfide bridges and aid in folding of transmembrane and secretory proteins in conjunction with protein disulfide isomerase (PDI)¹³⁻¹⁶. ERO1α molecular interactions and oxidation of PDI and protein folding has been well established^{17, 18}. While in its' reduced form ERO1α utilizes molecular oxygen, becoming oxidized, while reducing FAD through electron transfer and producing hydrogen peroxide as an intermediate. This process is important for the formation of disulfide bond formation that occurs during the protein folding process. Despite the cell being able to deal with minimal peroxide intermediates, it is also tightly regulated to avoid futile oxidative cycles from occurring in the ER, and therefore, aids in maintaining homeostasis within the ER¹⁹. ERO1α and PDI are the two main players in oxidative reactions occurring within the ER, both which are highly conserved from yeast to mammals¹³⁻¹⁶. Upon homeostatic imbalances within the endoplasmic reticulum, the unfolded protein response is generally activated; resulting in one of two possible outcomes: 1.) the unfolded protein response can return endoplasmic stress to homeostatic levels, or 2.) if homeostasis can't be established then apoptosis can be activated^{20, 21}. Thus, the unfolded protein response is a cellular response mechanism for resolving ER stress which can lead to survival or death depending on the context. Due to the critical enzymatic function, it is not surprising that ERO1α is tightly regulated via post-translational phosphorylation²², and through regulatory disulfide bridging. Cysteine bridging between Cys94-Cys99 allows for nucleophilic attack on bridged cysteines of Cys394-Cys397, allowing for Cys397 to undergo a nucleophilic attack onto bound FAD²³. This reaction process utilizes molecular oxygen as a terminal electron acceptor to form hydrogen peroxide as an intermediate. Cancer cells typically have increased ER stress compared to normal cells and occurs more so in secretory tumors such as lung, breast, multiple myeloma, and pancreatic cancers, but other tumors can exhibit high levels of ER stress following exposure to chemotherapy or hypoxia²⁴⁻²⁷. ERO1α plays a crucial role in ER homeostasis, via its' interaction with PDI to aid in protein folding, and has emerged as a key player in aiding in tolerance of cancer cells to ER stress²⁸. Despite the emerging evidence that ERO1α is an attractive target, few chemical probes are available to allow for further validation of the target. The challenge in part is due to the conserved nature of the FAD binding domain across enzymes. Developing specific inhibitors or more potent inhibitors to a specific flavoenzyme is critical for pharmacological validation of the target. Despite this challenge there are flavoenzyme targeting agents that are more specific such as the LSD-1 inhibitor IV. LSD-1 inhibitor IV forms a covalent adduct with FAD within LSD-1 and is approximately 6-fold more selective toward LSD-1 compared to MAO-A and MAO-B, and was originally described and synthesized by Neelamegam et al²⁹. The first reported ERO1α inhibitor was identified from a high-throughput screen. The hit molecule called EN-460 was shown to inhibit ERO1α activation and prevent re-oxidation³⁰. However, recently our laboratory demonstrated that EN-460 inhibits multiple flavoenzyme family members including LSD-1, MAO-A, and MAO-B⁶ and thus EN-460 is not an ideal compound for pharmacological credentialing of ERO1α. Due to this gap in knowledge, we sought to identify additional chemical space for targeting ERO1α.

The aurone class of compounds with sulfuretin as primary example, is a major group of flavonoid small molecules isolated from the heartwood of Rhus verniciflua and can be used to reduce oxidative stress³¹. Chalones are another class of flavonoids that exhibits a close biochemical relationship with the aurones³². Flavonoid compounds belong to the low-molecular-weight class of phenolic compounds that are widely distributed in the plant kingdom³³. Flavonoid compounds contain specific structural features being the 2-phenyl-benzol (α)pyrane, containing two benzene rings, linked by the heterocyclic pyrane ring³⁴. The main classes of flavonoids includes the aurones, chalcones, flavones, flavanones, isoflavones, anthocyanidins, flavan-3-ols, flavans, flavan-3,4-diols, dihydrochalcones, and proanthocyanidins^{35, 36}. Flavones are classified by the double bond at position 2 and 3 with a ketone at position 4 of the C ring while, most flavones have a hydroxyl group occurring at position 5 of the A ring³³. This invention provides certain aurone and/or chalcone analogs having activity toward ERO1α.

### SUMMARY OF THE INVENTION

In one aspect of this invention, compounds that are derivatives of sulfuretin are provided. The sulfuretin derivatives of this invention are compounds that are inhibitors for the endoplasmic reticulum oxidoreductin-1 alpha protein (ERO1α).

In certain embodiments of this invention, a compound is provided selected from one of the group consisting of:

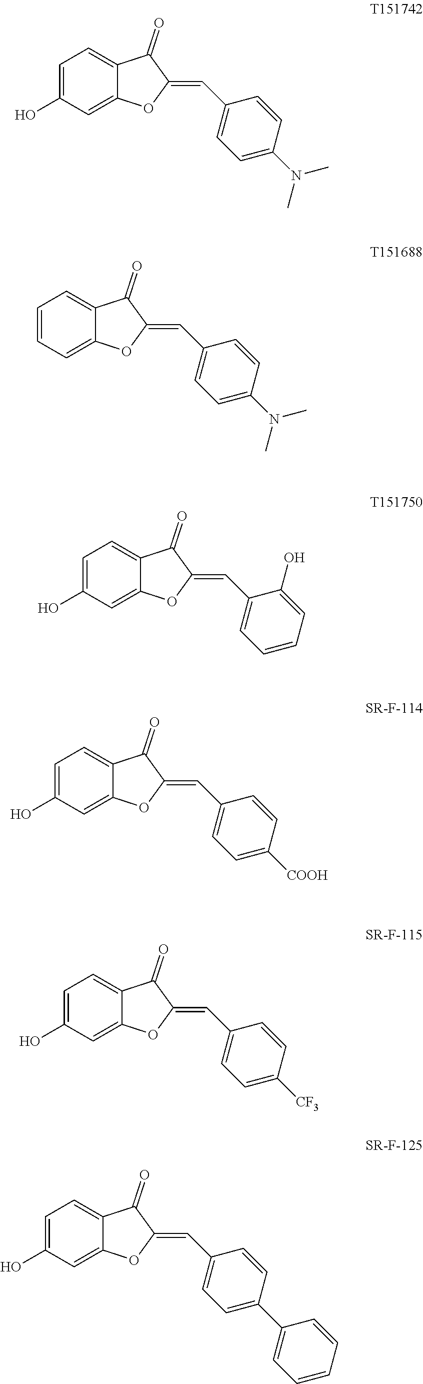 [SMILES: CN(C)c1ccc(/C=C2\Oc3cc(O)ccc3C2=O)cc1 ; CN(C)c1ccc(/C=C2\Oc3ccccc3C2=O)cc1 ; O=C1/C(=C/c2ccccc2O)Oc2cc(O)ccc21 ; O=C(O)c1ccc(/C=C2\Oc3cc(O)ccc3C2=O)cc1 ; O=C1/C(=C/c2ccc(C(F)(F)F)cc2)Oc2cc(O)ccc21 ; O=C1/C(=C/c2ccc(-c3ccccc3)cc2)Oc2cc(O)ccc21]

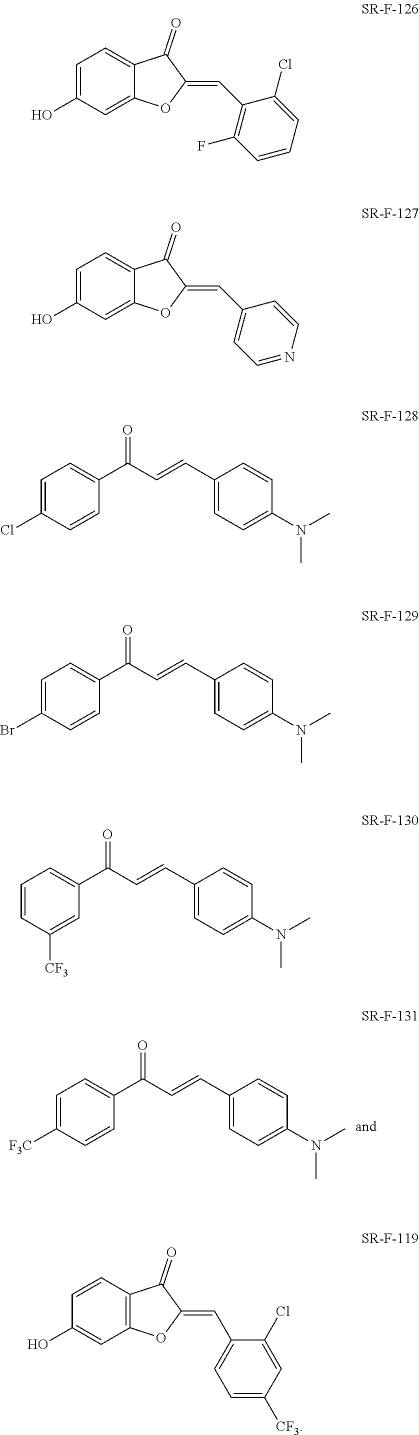

In another embodiment of this invention, a compound is provided that is selected from one of the group consisting of:

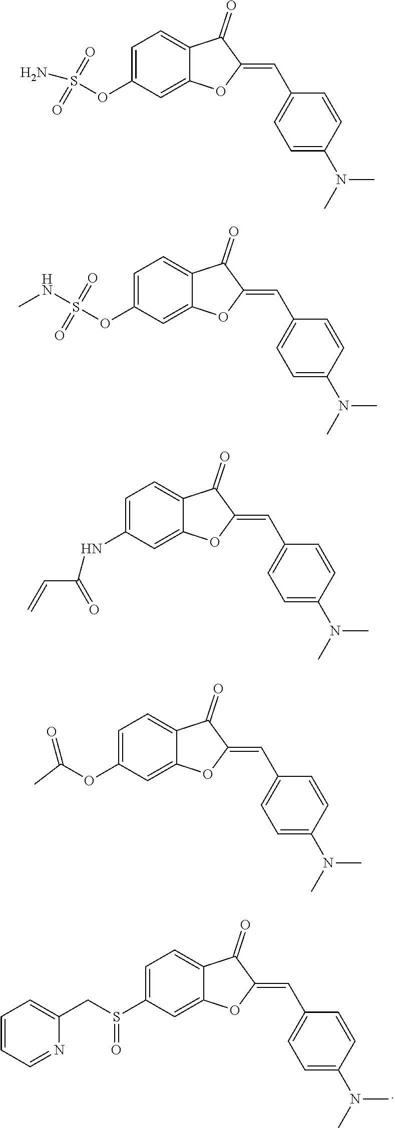

In another embodiment of this invention, a compound is provided that is selected from one of the group consisting of:

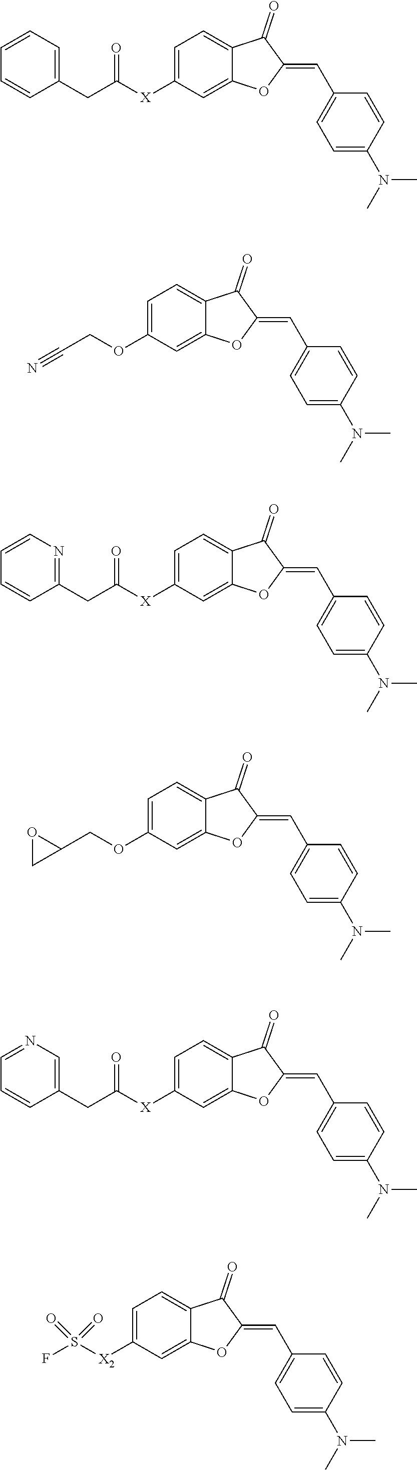

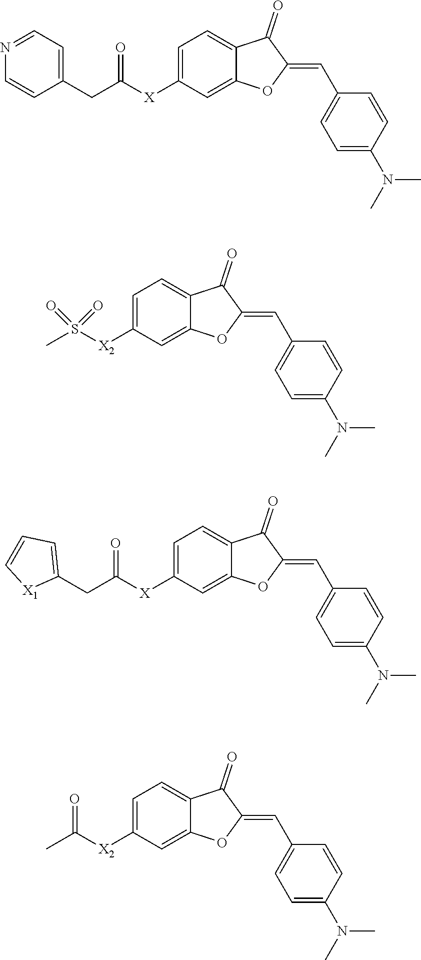

wherein X=O, S, NH, or NMe; X₁=O, S, NH, or NMe; and X₂=O, NH, or NMe.

Pharmaceutical compositions having these compounds, and pharmaceutical compositions having these compounds and one or more pharmaceutically acceptable carrier(s) are provided.

In another aspect of this invention, a method is provided of targeting Ero1α for treatment of cancer. In a certain aspect of this invention, the cancer is lung cancer. The lung cancer may be non-small cell lung cancer.

### BRIEF DESCRIPTION OF THE DRAWINGS

FIG. 1 shows a graph that sets forth that T151742 is the most potent aurone and is two-fold more potent compared to EN-460.

FIG. 2 shows the structures of known EN-460 and sulfuretin and the structures of the compounds of the instant invention T151742, T151750, T151688, SR-F-114, SR-F-115, SR-F-125, SR-F-126, SR-F-127, SR-F-128, SR-F-129, SR-F-130, and SR-F-131.

FIG. 3 shows the electrostatic surfaces of certain compounds of this invention.

FIG. 4A shows enzymatic activity of purified human ERO1α (0.0625 mg/mL), varying concentrations of purified human PDI (0.25 μM, 0.125 μM, 0.0625 μM, 0.03125 μM, and 0.5% DMSO), and varying concentrations of T151742 (100 μM, 75 μM, 50 μM, 25 μM, 10 μM, 0.1 μM and 0.5% DMSOB.)

FIG. 4B shows a Lineweaver-Burk plot produced from taking the double reciprocal (1/[PDI]) vs (1/[T151742]).

FIG. 4C shows a Surface Plasma Resonance that demonstrates binding of T151742 directly to ERO1α (Kd=31.4 μM) in a concentration dependent manner

FIG. 5A shows a docking study wherein compound T151742 is docked with optimal hydrogen bonding of the 6-hydroxyl to GLN93 and the 3-ketone to GLY138.

FIG. 5B shows a docking study wherein loss of the 6-hydroxyl in compound T151688 led to a decrease in binding.

FIG. 5C shows a docking study wherein loss of the 4-dimethylamino leads to reposing with GLY138 bond not forming compared to compound T151742.

FIG. 6A shows a vertical row of graphs wherein PC-9 Cells were subjected to a cellular thermal shift assay in the absence or presence of T151742. Upon addition of T151742, Tagg was quantified for ERO1α (top graph FIG. 6A), ERO1β (middle graph, FIG. 6A), and LSD-1 (bottom graph, FIG. 6A).

FIG. 6B shows a MTT viability assay comparing potency between ERO1α inhibitor EN-460, T151742, and T151742 derivative SR-F-114, revealing SR-F-114 is about two-fold less effective in vitro versus T151742 and EN-460 in the PC-9 non-small cell lung cancer cell line.

FIG. 6C shows a graph that sets forth a validation of ERO1α knockout in the PC-9 cell line utilizing CRISPR.

FIG. 6D shows two CRISPR deleted ERO1α clones that were subjected to treatment of T151742 as well as the non-targeted control PC-9 cell line.

FIG. 7A shows soft agar clonogenic assays demonstrates T151742 is more potent compared to EN-460, wherein 1000 cells/well were plated in a 12-well plate in triplicate with either 0.5% DMSO, T151742, or EN-460 and allowed to form colonies for 14 days.

FIG. 7B shows that the colonies of FIG. 7A were subsequently stained with 0.01% Crystal Violet, imaged, and quantified using ImageJ, with 0.5% DMSO (far left bar, FIG. 7B), 3.75 μM (second bar from left), 7.5 μM (third bar from left), and 15 μM (fourth bar from left).

FIG. 7C shows soft agar clonogenic assays of two ERO1α knockout clones that are resistant to T151742 treatment, wherein 1500 cells/well were plated in a 12-well in triplicate with either 0.5% DMSO or T151742 and allowed to form colonies for 14 days.

FIG. 7D shows the colonies of FIG. 7C were stained with 0.01% Crystal Violet, imaged, and quantified using ImageJ, with 0.05% DMSO (far left bar, FIG. 7D), 3.75 μM T151742 (second bar from left), 7.5 μM T151742 (third bar from left), and 15 μM T151742 (fourth bar from left).

FIG. 8 shows that the LSD1 inhibitor IV and tranylcypromine both known to covalently crosslink FAD did not inhibit ERO1α activity.

FIG. 9 shows that T151742 does not inhibit LSD-1 enzymatic activity.

FIG. 10 shows structures of compounds of this invention.

FIG. 11 shows structures of compounds of this invention.

### DETAILED DESCRIPTION OF THE INVENTION

This invention provides various compounds that are derivatives of sulfuretin. The compounds that are sulfuretin derivatives of this invention are inhibitors for the endoplasmic reticulum oxidoreductin-1α protein.

In another aspect of this invention, a method is provided of targeting Ero1α for treatment of cancer. The cancer may be lung cancer. The lung cancer may be non-small cell lung cancer.

In certain aspects of this invention, the compounds have chemical structures described herein as T151742, T151750, T151688, SR-F-153, SR-F-154, SR-F-158, SR-F-160, SR-F-114, SR-F-115, SR-F-119, and SR-F-125-131, and T151747-SO2F. These compounds are inhibitors of the endoplasmic reticulum oxidoreductin-1 alpha protein. These compounds are selected from one of the groups consisting of:

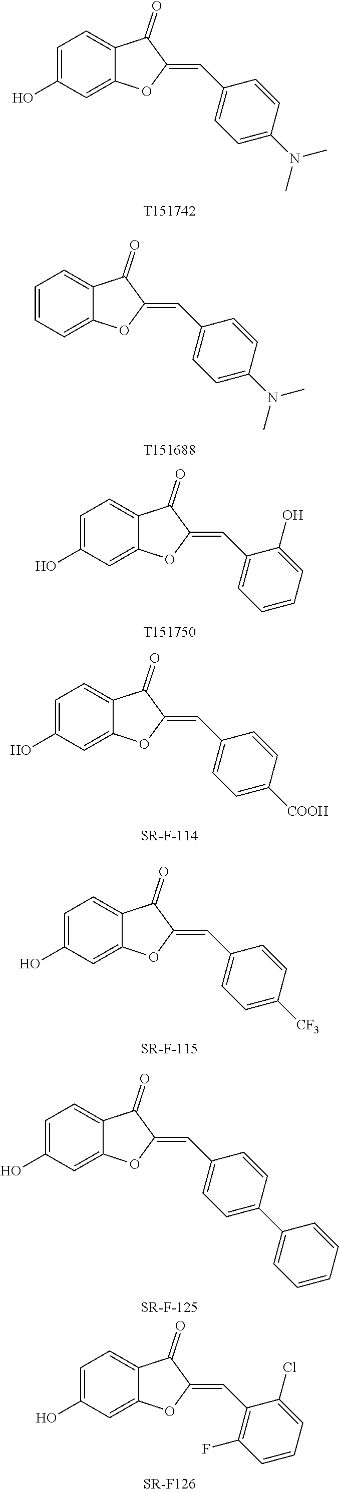

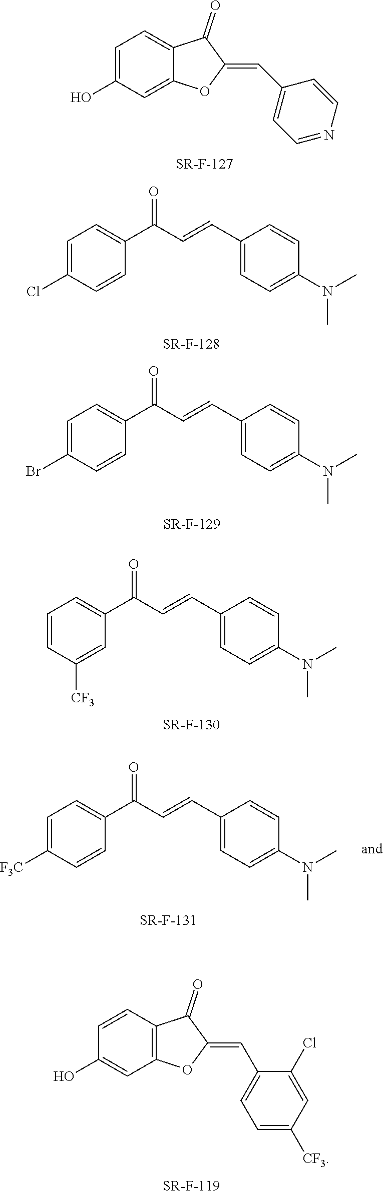 [SMILES: O=C1/C(=C/c2ccncc2)Oc2cc(O)ccc21 ; CN(C)c1ccc(/C=C/C(=O)c2ccc(Cl)cc2)cc1 ; CN(C)c1ccc(/C=C/C(=O)c2ccc(Br)cc2)cc1 ; CN(C)c1ccc(/C=C/C(=O)c2cccc(C(F)(F)F)c2)cc1 ; CN(C)c1ccc(/C=C/C(=O)c2ccc(C(F)(F)F)cc2)cc1 ; O=C1/C(=C/c2ccc(C(F)(F)F)cc2Cl)Oc2cc(O)ccc21]

In another embodiment of this invention, a pharmaceutical composition is provided comprising a compound selected from one of the group consisting of:

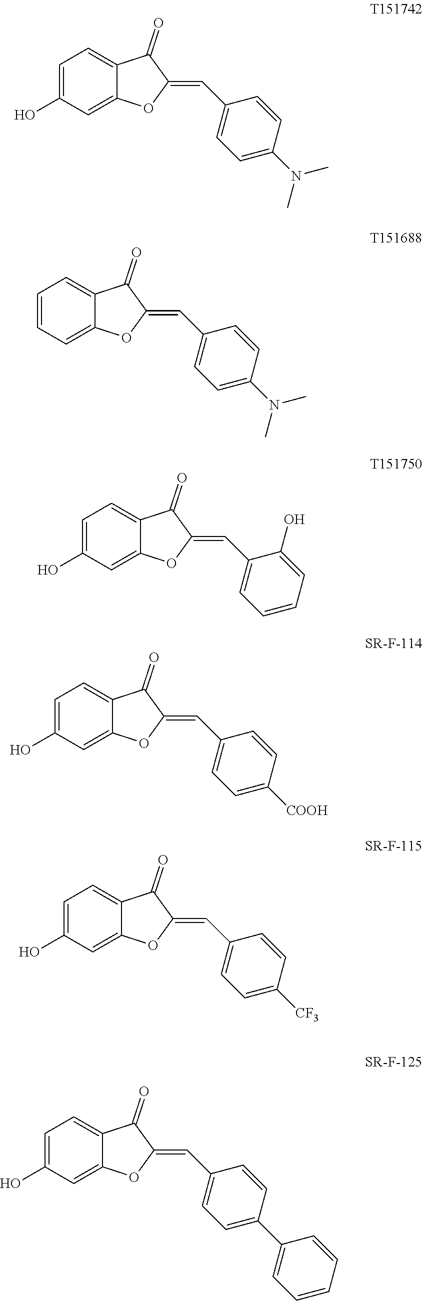 [SMILES: CN(C)c1ccc(/C=C2\Oc3cc(O)ccc3C2=O)cc1 ; CN(C)c1ccc(/C=C2\Oc3ccccc3C2=O)cc1 ; O=C1/C(=C/c2ccccc2O)Oc2cc(O)ccc21 ; O=C(O)c1ccc(/C=C2\Oc3cc(O)ccc3C2=O)cc1 ; O=C1/C(=C/c2ccc(C(F)(F)F)cc2)Oc2cc(O)ccc21 ; O=C1/C(=C/c2ccc(-c3ccccc3)cc2)Oc2cc(O)ccc21]

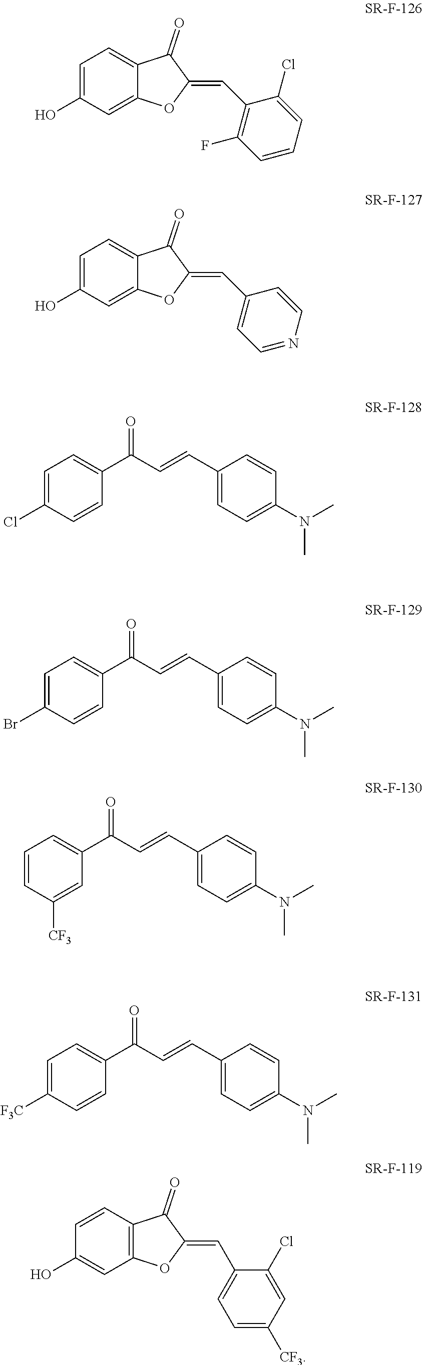

In another embodiment of this invention, a pharmaceutical composition is provided comprising a compound selected from one of the groups consisting of:

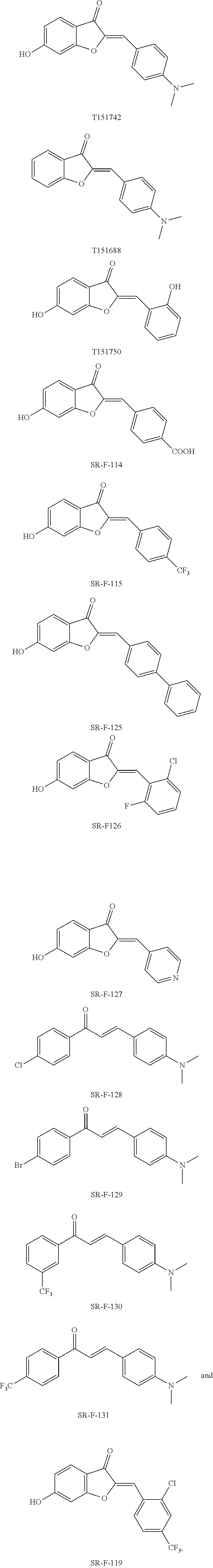

and
a pharmaceutically acceptable carrier.

In another embodiment of this invention, a compound is provided selected from one of the groups consisting of:

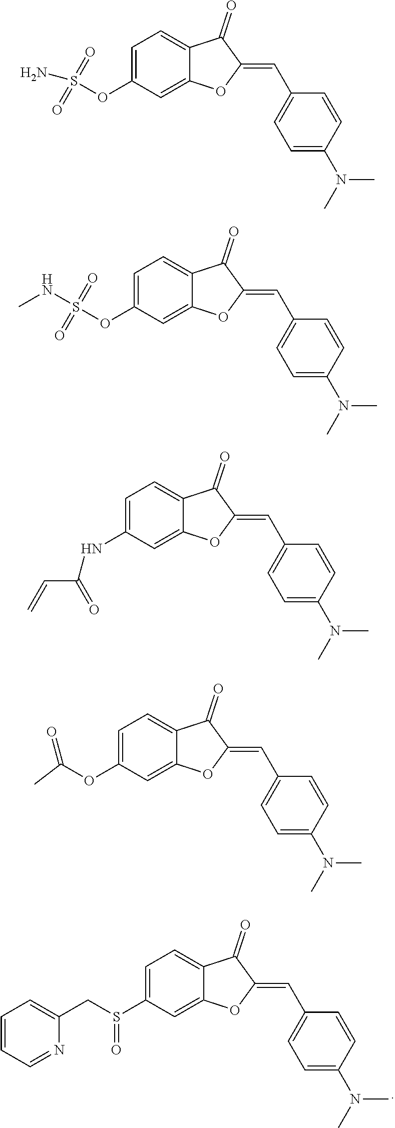

In another embodiment of this invention, a pharmaceutical composition is provided comprising a compound selected from one of the groups consisting of:

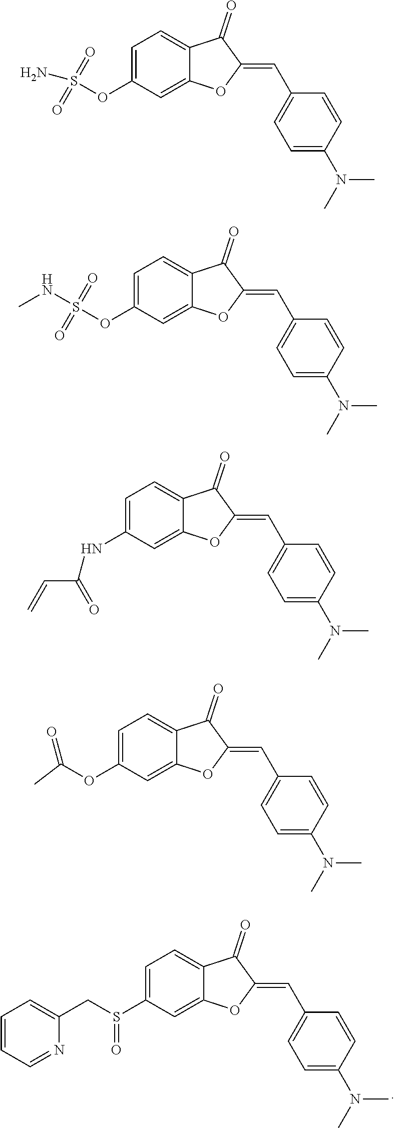

In another embodiment of this invention, a pharmaceutical composition comprising a compound selected from one of the groups consisting of:

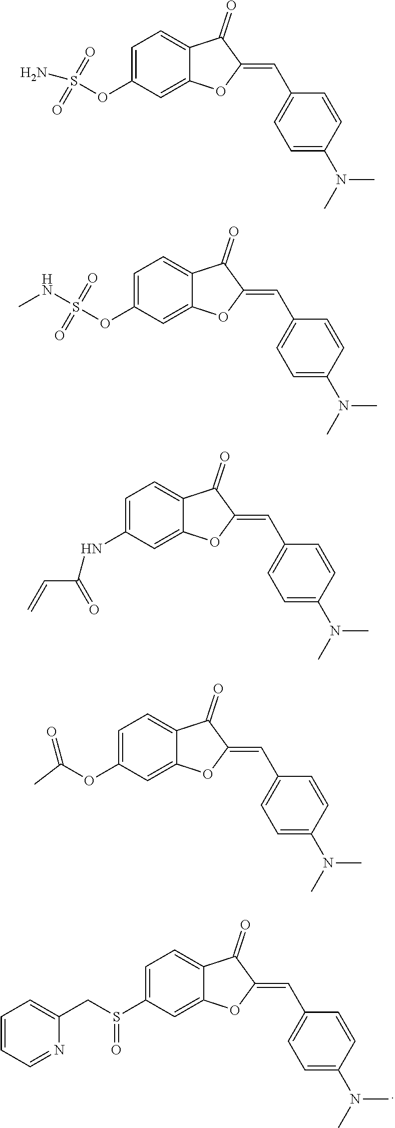

and a pharmaceutically acceptable carrier.

In another embodiment of this invention, a compound is provided that is one selected from the groups consisting of:

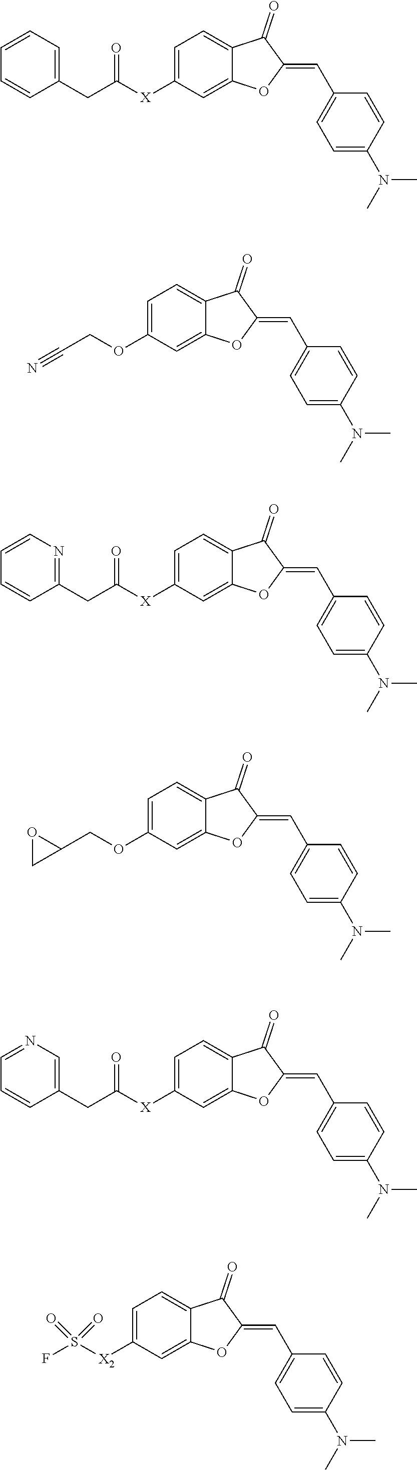

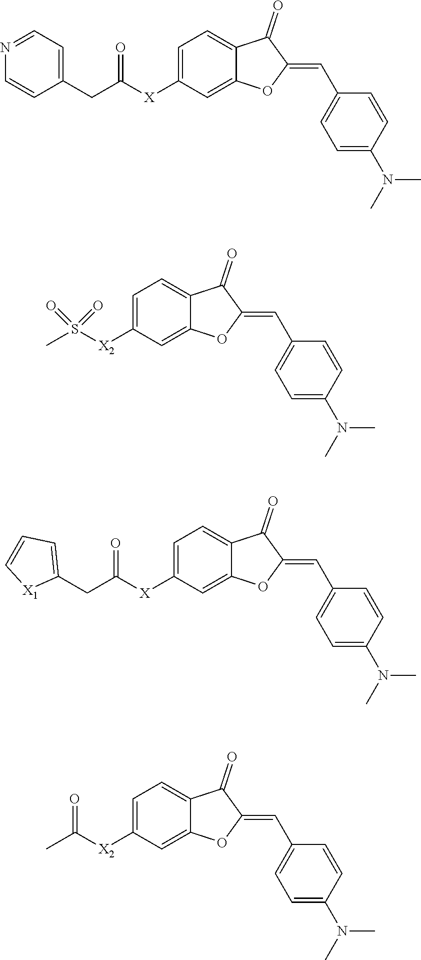

wherein X=O, S, NH, or NMe; X₁=O, S, NH, or NMe; and X₂=O, NH, or NMe.

In another embodiment of this invention, a pharmaceutical composition is provided comprising at least one compound selected from the groups consisting of:

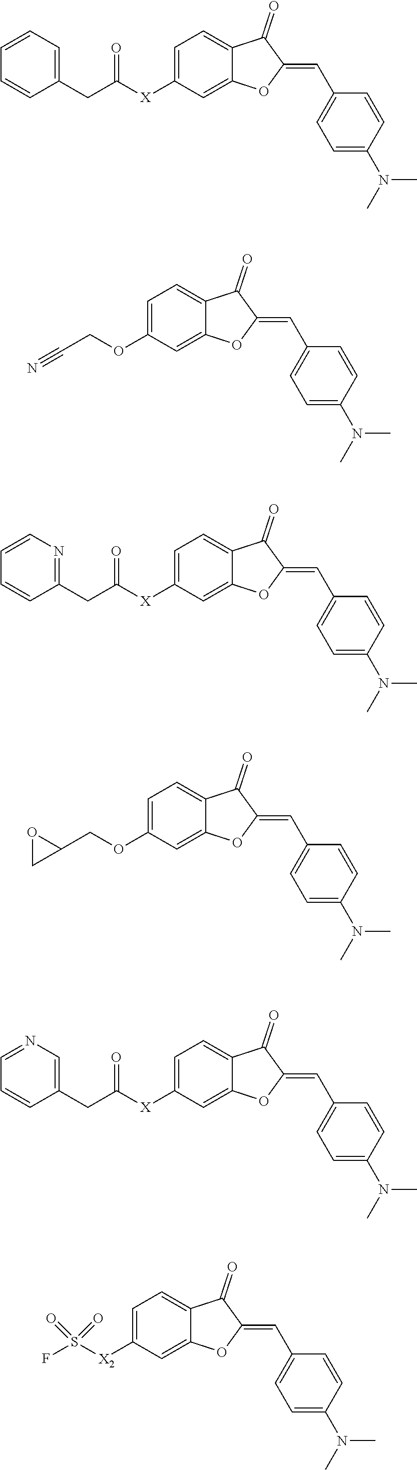

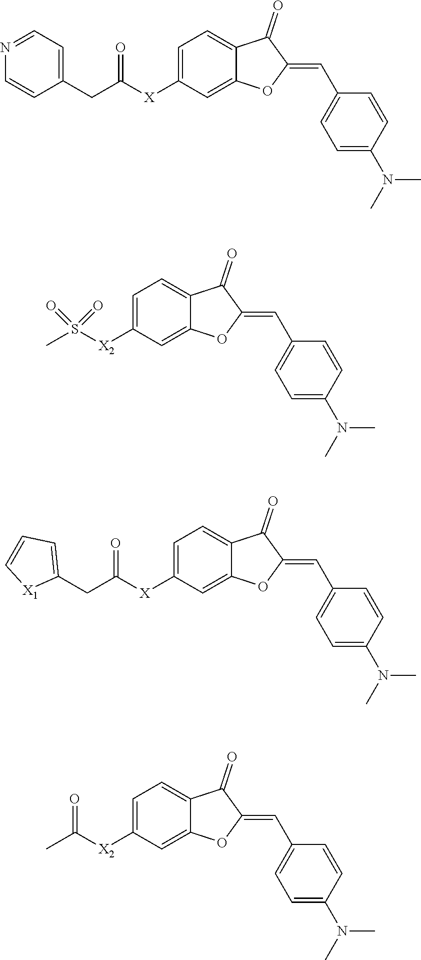

wherein X=O, S, NH, or NMe; X₁=O, S, NH, or NMe; and X₂=O, NH, or NMe.

In another embodiment of this invention, a pharmaceutical composition is provided comprising a compound that is one selected from the groups consisting of:

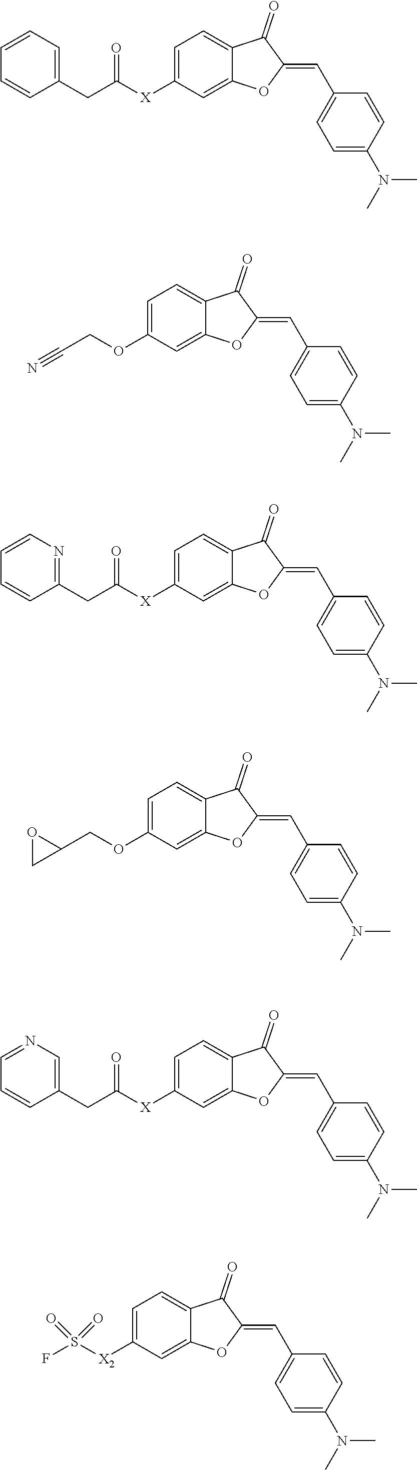

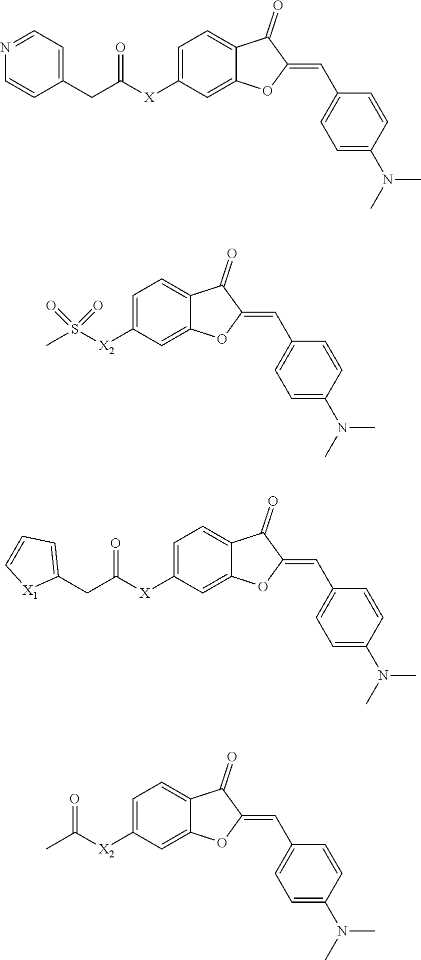

wherein X=O, S, NH, or NMe; X₁=O, S, NH, or NMe; and X₂=O, NH, or NMe, and a pharmaceutically acceptable carrier.

In another aspect of this invention, a pharmaceutical composition is provided comprising a compound that is a derivative of sulfuretin and a pharmaceutically acceptable carrier.

As used herein, the term “patient” means members of the animal kingdom, including, but not limited to human beings.

As used herein, the term “effective amount” or “therapeutically effective amount” refers to that amount of the present compounds, and salts thereof, and/or compositions required to bring about a desired effect in a patient. The desired effect will vary depending upon the illness or disease state being treated. For example, the desired effect may be reducing the tumor size, destroying cancerous cells, and/or preventing metastasis, and one of which may be the desired therapeutic response. On its most basic level, a therapeutically effective amount is that amount of a substance that is needed to inhibit mitosis of a cancerous cell.

As used herein, an “acceptable pharmaceutical carrier” refers to any pharmaceutical carrier known in the art, absent compatibility problems with the novel compounds of this invention. Generally, the pharmaceutical carrier includes, for example, but not limited to, physiologic saline, 5% dextrose in water, combinations thereof, lactose, sucrose, gelatin, and polymers.

It is well within the skill of one practicing in the art, a therapeutically effective amount of the compounds of this invention may be administered by any means known in the art, including but not limited to, injection, parenterally, intravenously, intraperitoneally, orally, rectally, and topically.

The compounds of this invention or pharmaceutically acceptable salts or hydrates of the compounds of this invention, can be incorporated into, for example but not limited to, tablets, capsules, elixirs, suspensions, solutions, syrups, and sustained-release preparations and formulations. The compounds of this invention may be incorporated with, for example but not limited to, binders, excipients, disintegrating agents, lubricants, sweetening agents, preservatives, dyes, and flavoring agents.

In another aspect of this invention, a method of treating a patient having cancer comprising administering to said patient a therapeutically effective amount of a compound that is a derivative of sulfuretin to a patient is provided. In certain embodiments of this invention, the compound has a chemical structure of T151742, T151750, T151688, SR-F-153, SR-F-154, SR-F-158, SR-F-160, SR-F-114, SR-F-115, SR-F-119, and SR-F-125-131, and T151747-SO2F, as shown herein. This method includes wherein the compound that is a derivative of sulfuretin targets Ero1α for treatment of said patient having cancer. The cancer may be lung cancer. The lung cancer may be non-small cell lung cancer.

The flavin adenine dinucleotide containing Endoplasmic Reticulum Oxidoreductase-1α (ERO1α) catalyzes the formation of de novo disulfide bond formation of secretory and transmembrane proteins and contributes towards proper protein folding. Recently, increased ERO1α expression has been shown to contribute to increased tumor growth and metastasis in multiple cancer types. In this report we sought to define novel chemical space for targeting ERO1α function. Using the previously reported ERO1α inhibitor compound, EN-460, as a benchmark pharmacological tool we were able to identify a sulfuretin derivative, T151742 which was approximately two-fold more potent using a recombinant enzyme assay system (IC₅₀=8.27±2.33 μM) compared to EN-460 (IC₅₀=16.46±3.47 μM). Additionally, T151742 (IC₅₀=16.04 μM) was slightly more sensitive than EN-460 (IC₅₀=19.35 μM) using an MTT assay as an endpoint. Utilizing a cellular thermal shift assay (CETSA), we determined that the sulfuretin derivative T151742 demonstrated isozyme specificity for ERO1α as compared to ERO1β and showed no detectable binding to the FAD containing enzyme LSD-1. T151742 retained activity in PC-9 cells in a clonogenicity assay while EN-460 was devoid of activity. Furthermore, the activity of T151742 inhibition of clonogenicity was dependent on ERO1α expression as CRISPR edited PC-9 cells were resistant to treatment with T151742. In summary we identified a new scaffold that shows specificity for ERO1α compared to the closely related paralog ERO1β or the FAD containing enzyme LSD-1 that can be used as a tool compound for inhibition of ERO1α to allow for pharmacological validation of the role of ERO1α in cancer.

### Results and Discussions:

### Determination of Inhibition of ERO1α by Sulfuretin Derivatives

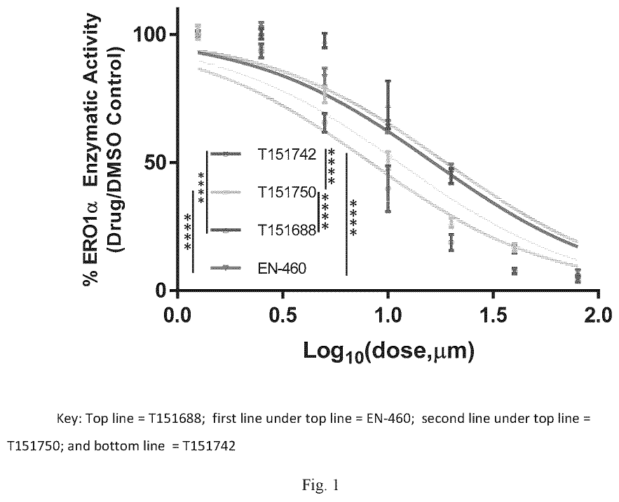

Based on previous work which established an aurone chemical class of compounds as FAD containing enzyme inhibitors³⁷, we evaluated sulfuretin analogs for ERO1α enzyme inhibition. The Amplex Red assay was used to determine if the sulfuretin derivatives were capable of inhibition of ERO1α in a recombinant assay system previously established by our laboratory⁶. As shown in FIG. 1 (FIG. 1), the sulfuretin derivatives inhibited ERO1α in a concentration dependent manner. The most potent aurone analog referred to as T151742 was found to be approximately 2-fold more potent compared to EN-460 (IC₅₀=8.27±2.33 μM), T151750 (IC₅₀=11.34±2.49 μM), T151688 (IC₅₀=18.91±5.47 μM) and EN-460 (IC₅₀=16.46±3.47 μM).

FIG. 1 shows that T151742 is the most potent aurone and is two-fold more potent compared to EN-460. Purified human ERO1α (0.0625 mg/mL) and purified human PDI (0.0625 mg/mL) were incubated with increasing concentrations of EN-460, T151742, T151688 or T151750. Each data point shown is the average ±S.D (N=3 independent experiments and performed in triplicates). ****p<0.0001 by Two-way ANOVA.

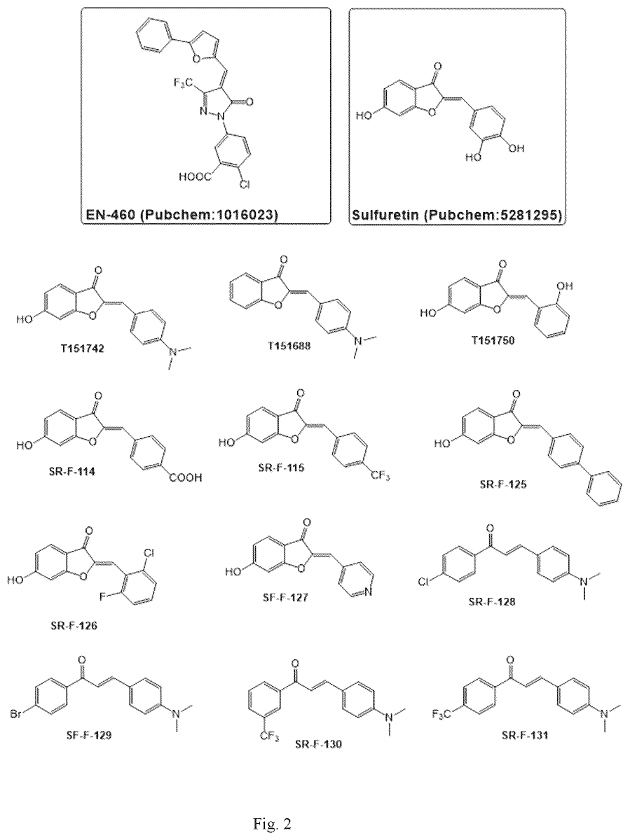

Based on our initial studies shown in FIG. 1, we sought to determine whether the 4-dimethylamino on the benzylidene ring was critical for inhibition of ERO1α and synthesized a small set of compounds to determine the impact of substitution on the benzylidene ring has on enzyme activity. The compound structures are shown in FIG. 2 that were evaluated for ERO1α enzyme activity. Table 1 shows the compounds of FIG. 2 that were tested at 10 μM which is the approximate IC₅₀ of T151742.

TABLE 1
Inhibition of ERO1α enzyme activity, reported
as percent inhibition compared to vehicle control.
Shown is the average of N = 3 ± S.D.

| Compound | % inhibition (10 μM) | Classification |
|---|---|---|
| T151742 | 55.31 ± 0.98 | Aurone |
| T151688 | 40.46 ± 7.32 | Aurone |
| T151750 | 48.60 ± 4.68 | Aurone |
| SR-F-114 | 28.62 ± 5.57 | Aurone |
| SR-F-115 | 27.48 ± 1.82. | Aurone |
| SR-F-125 | 20.73 ± 4.92 | Aurone |
| SR-F-126 | 38.74 ± 1.56 | Aurone |
| SR-F-127 | 33.40 ± 2.72 | Aurone |
| SR-F-128 | 37.86 ± 2.42 | Chalcone |
| SR-F-129 | 26.75 ± 1.44 | Chalcone |
| SR-F-130 | 27.26 ± 3.16 | Chalcone |
| SR-F-131 | 24.16 ± 0.52 | Chalcone |

% Inhibition for each compound at 10 μM was determined by recombinant enzyme assay as previously described⁶

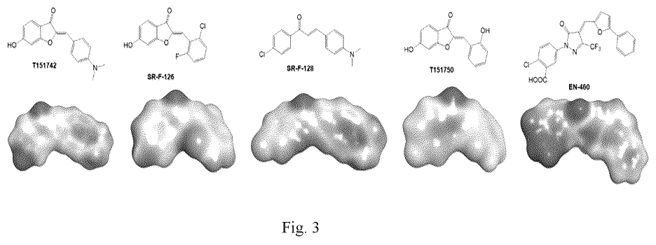

The sulfuretin derivatives were found to inhibit ERO1α, with the Aurone scaffolds demonstrating similar activity to the chalcone compounds (Table 1). With the small changes in activity profile, our findings suggest that the 4-dimethylamino-moiety to be important for activity, since none of the new aurones or chalcones were more potent than T151742. Additionally, the para-hydroxy on the benzofuran is important for activity, as loss of this functional group led to a decrease in inhibition activity. Comparing the electrostatic surfaces of EN-460, to the compounds tested here, we found that interestingly, T151742 shared a similar surface pattern as EN-460, while other compounds such as SR-F-126, SR-F-128, and T151750 shared some overlap in electrostatics, but were less pronounced and concentrated in certain areas, as compared to EN-460 (FIG. 3). This in part could explain the differences in activity profile of the compounds compared to T151742, and EN-460.

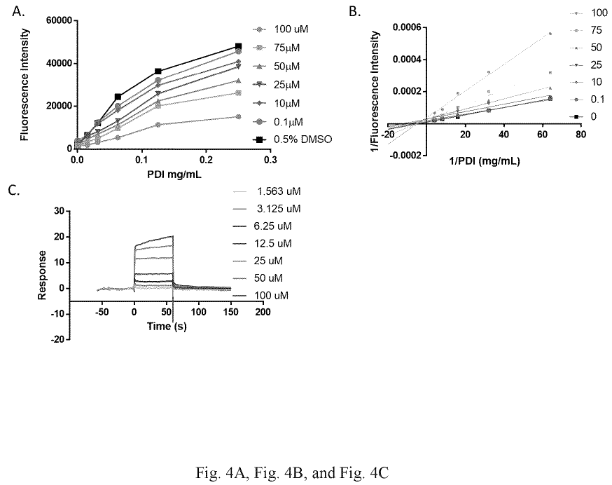

FIG. 3 shows the electrostatic surfaces of the compounds of this invention. Surface-electrostatic potential differences are noticeable on the aurone and Chalcone derivatives as compared to the ERO1α inhibitor EN-460, with T151742 showing stronger correlation. Blue shows positive charge areas, and red shows negative charge areas. Since the newly identified sulfuretin derivative T151742 was the most potent of the compounds tested we evaluated its activity profile in classical enzyme kinetics assays. Currently, the standard model of FAD-containing enzymes, is that the inhibitors available inhibit flavoenzymes interaction directly with FAD substrate pocket and are competitive inhibitors, with a select set of compounds designed as covalent inhibitors³⁸. To validate the mechanism of inhibition T151742 had on ERO1α, varying PDI concentrations (the substrate for ERO1α), and varying concentrations of T151742 were used and the double reciprocal of this plot was taken to produce the Lineweaver-Burk plot (See FIG. 4A and FIG. 4B). As shown in FIG. 4A and FIG. 4B, K_{M} is increased while V_{Max} is reduced with the increasing concentration of T151742. This is observed in the double-reciprocal plot in FIG. 4B, as slope of the lines increase in a concentration dependent manner, and both x and y-intercepts change in a concentration dependent manner as well. These findings suggest that T151742 was acting upon ERO1α as a non-competitive (mixed) inhibitor and was not binding directly to the active site.

Surface plasma resonance (SPR) was utilized as an orthogonal assay to confirm direct binding between T151742 and ERO1α. For the SPR assay, recombinant ERO1α was crosslinked to a C5 chip and varying concentrations of ligand T151742 were evaluated for detection of changes in refractive index, due to change in mass on the chip when ligand is bound to the protein. As shown in FIG. 4C, T15742 binds to ERO1α in a concentration dependent manner with a Kd=31.4 μM. The relatively high Kd value is in part due to the fast off-rate. Taken together, our data indicate that the sulfuretin derivative T151742 i) binds directly to ERO1α in a recombinant system ii) is the most potent analog in this small series at inhibiting ERO1α, and two-fold more potent than EN-460, iii) acts as a non-competitive (mixed) inhibitor and iv) a remaining potential liability is the fast off rate of the compound.

FIG. 4A shows that T151742 binds directly to ERO1α and acts as a non-competitive (mixed) inhibitor. FIG. 4A shows purified human ERO1α (0.0625 mg/mL), varying concentrations of purified human PDI (0.25 μM, 0.125 μM, 0.0625 μM, 0.03125 μM, and 0.5% DMSO), and varying concentrations of T151742 (100 μM, 75 μM, 50 μM, 25 μM, 10 μM, 0.1 μM and 0.5% DMSO) were assayed using Amplex Red assay to quantify enzymatic activity. FIG. 4B shows a Lineweaver-Burk plot produced from taking the double reciprocal (1/[PDI]) vs (1/[T151742]). FIG. 4C shows a Surface Plasma Resonance that demonstrates binding of T151742 directly to ERO1α (Kd=31.4 μM).

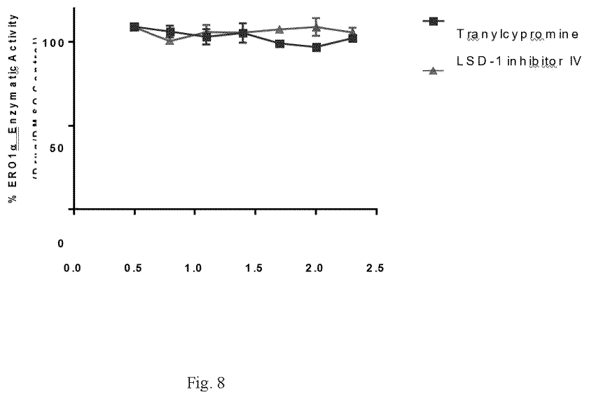

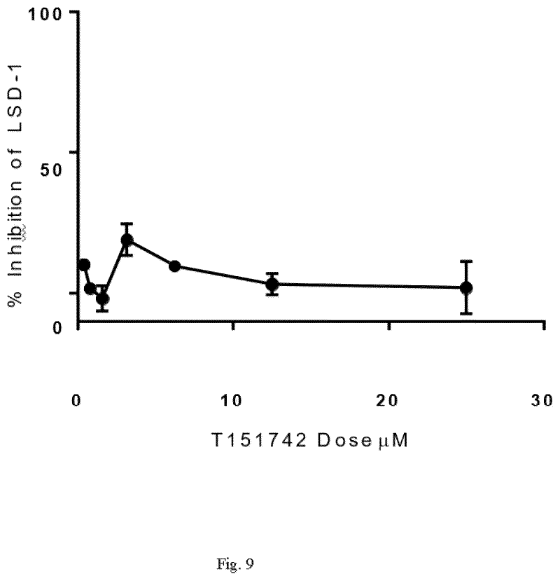

Several of the flavoenzyme family of proteins contain a substrate pocket in which specific substrates are metabolized in the presence of FAD, e.g., dopamine with MAO-A³⁹. This has led to the development of irreversible covalent inhibitors of the cofactor FAD such as tranylcypromine⁴⁰. The development of the covalent inhibition led to the inclusion of the active moiety propargylamine, leading to derivatives deprenyl/selegiline and rasagiline which are used clinically to treat Parkinson's disease⁴¹. Furthermore, the LSD-1 inhibitor IV, RN-1 was amongst the first lysine demethylase inhibitors discovered, where LSD-1 is a FAD containing enzyme²⁹. Tranylcypromine forms a covalent modification directly with FAD⁴⁰. LSD-1 inhibitor IV, RN-1 contains the classical tranylcypromine-template that allows for covalent modification of targeted enzymes containing a FAD moiety. We determined that the LSD1 inhibitor IV and tranylcypromine both known to covalently crosslink FAD did not inhibit ERO1α activity (See FIG. 8, for concentration response). Conversely, T151742 was shown not to inhibit LSD-1 enzymatic activity (See FIG. 9 for concentration response). These data suggest that the FAD containing ring within ERO1α is concealed within the enzyme and not easily targeted by small molecules. Based on the crystal structure of ERO1α, (3ahq.pdb), the FAD is contained in a pocket close to the redox active cysteines, fully occupying the pocket space. Taken together, these findings suggest that the putative binding pocket of the ERO1α inhibitors are not in the FAD pocket but is likely allosteric to the FAD binding pocket.

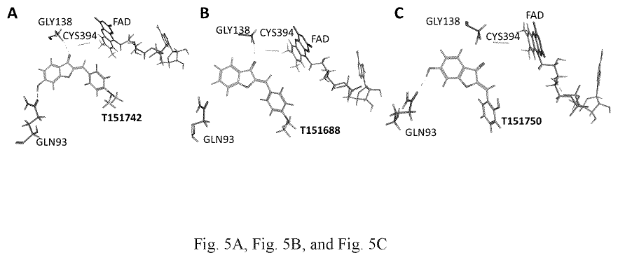

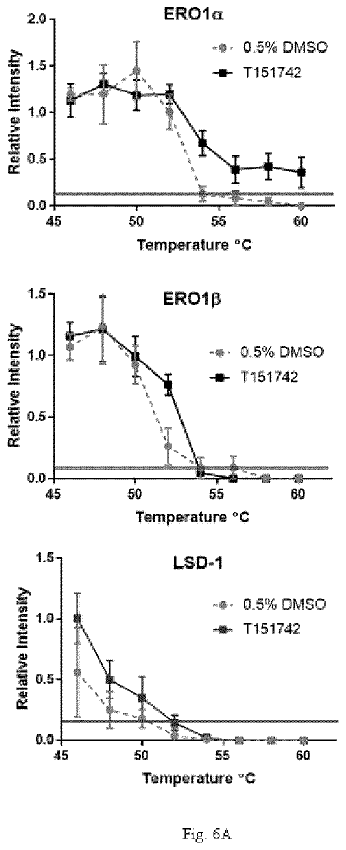

FIG. 5A, FIG. 5B, and Fig. C show docking studies of ERO1a inhibitors. The crystal structure of the active ERO1α (3ahq.pdb) with the co-factor FAD was used for binding studies. FIG. 5A shows T151742 docked with optimal hydrogen bonding of the 6-hydroxyl to GLN93 and the 3-ketone to GLY138. FIG. 5B shows a loss of the 6-hydroxyl in T151688 led to a decrease in binding. FIG. 5C shows a loss of the 4-dimethylamino leads to reposing with GLY138 bond not forming compared to T151742.

Based on the experimental data we explored binding pockets allosteric to the FAD binding pocket of ERO1α. The Site Finder algorithm of MOE 2020 was used to identify the putative pocket in proximity to the FAD binding pocket, as well as the reactive cysteines CYS394 and CYS397 (FIG. 5A). We used the reduced form of the crystal, based on our enzyme assay which still produced hydrogen peroxide in presence of the inhibitors. As shown in FIG. 5A, T151742 was predicted to bind by hydrogen bonding to GLN93 and GLY138 with the 3-ketone oriented towards the reactive cysteines CYS394 and CYS397 (<4 A). FIG. 5B shows that loss of the 6-hydroxyl moiety which yielded T151688 lead to a loss of the GLN93 binding interaction, potentially contributing to differences in activity when comparing T151742 with T151688. Predicted affinity using YASARA Autodock Vina corroborated these findings, with a predicted inhibition of 2.31 μM for T151742 and 13.67 μM for T151688. Lastly, FIG. 5C shows that loss of the 4-dimethylamino in T151750 leads to loss of the 3-ketone hydrogen bond with GLY138 as compared to T151742. From these docking studies, we propose that the ERO1α inhibitors are interacting with ERO1α via an allosteric binding site located in proximity to the redox active CYS394-CYS397 pair, preventing the oxidation of the disulfide bond.

### Determining the Selectivity of T151742 Using In Vitro Cell Culture Models

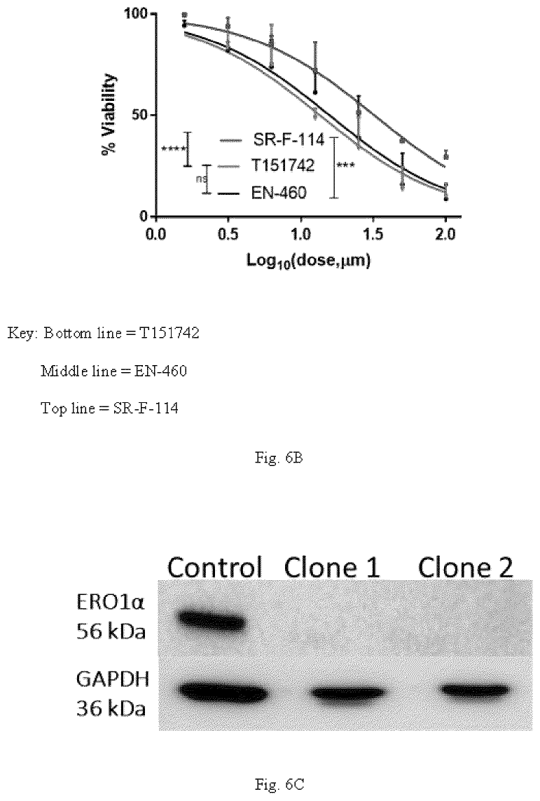

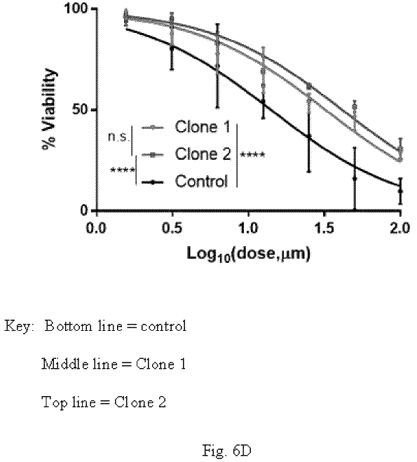

The ERO1α inhibitor EN-460 found in a high-throughput screen from natural products was shown to inhibit the reduced form of ERO1α³⁰. However, this compound has a liability as a tool to pharmacologically validate ERO1α as a target as it has been shown to inhibit other flavoenzyme family members including; LSD-1 MAO-A, and MAO-B^{6, 30}. It was previously shown that T151742 showed activity against MAO-A and MAO-B, albeit was not the most potent Aurone member³⁷. To date activity of T151742 towards other flavoenzymes has not been identified at this time. We utilized the Cellular Thermal Shift Assay (CETSA) to determine target engagement of T151742 in vitro using the non-small cell lung cancer (NSCLC) cell line PC-9. The EGFR driven PC-9 cell line was chosen as new treatment strategies are needed in EGFR driven NSCLC as most patients relapse and become resistant to current targeted therapies with development of further mutations such as T790M, C797S as well as emergence of EGFR independent tumors^{42, 43}. CETSA utilizes thermodynamic principles that have been well described and upon binding of ligands to a protein, the ligand bound protein becomes more thermo-stable with respect to the temperature required to denature the protein or expose hydrophobic regions of the protein leading to aggregation and insolubility of the protein⁴⁴. All proteins have an aggregation temperature which is inherent to their intracellular thermostability. Upon addition of a ligand binding, the thermostability increases. First, we determined the aggregation temperature (Tagg) of ERO1α, ERO1β, and LSD-1, which are all FAD dependent enzymes and potential in vitro targets of T15742. Tagg was defined as the point at which 10% of protein or less is remaining when compared to the room temperature control of the target protein by western blot analysis. As shown in FIG. 6A, CETSA analysis determined that the Tagg for unbound target protein (ERO1α Tagg=54° C.; ERO1β; Tagg=54° C.; and LSD-1 Tagg=52° C.). The values reported are averages from three independent experiments. When T151742 was added to the culture media at a concentration of 100 μM for 1 hour at 37° C. the Tagg for ERO1α shifted approximately ˜6 degrees to >60° C. (See FIG. 6A). In contrast, incubation of cells with 100 μM T151742 did not change the Tagg for LSD-1 or ERO1β. These results indicate that T151742 bound and stabilized ERO1α compared to the flavoenzymes ERO1β and LSD1. Although ERO1α and ERO1β, have highly conserved sequences and functions it appears their tertiary structures are more diverse than previously thought and specific inhibitors can be developed to distinguish the activity of inhibiting one enzyme using in vitro models. The Tagg for LSD-1 also remained unchanged upon addition of T151742 and aided in confirming that T151742 shows more specificity towards ERO1α as compared to other members of the flavoenzyme family, with the exception of MAO-A which was previously reported³⁷. The results of the CETSA assay combined with the in vitro recombinant assay demonstrated that T151742 does not inhibit enzymatic activity of LSD-1 (See FIG. 9 for concentration response).

FIG. 6A shows CETSA assay validates target engagement of T151742 towards ERO1α and proliferation is partially dependent on ERO1α expression by MTT assay in PC-9 cells. FIG. 6A shows PC-9 Cells were subjected to the cellular thermal shift assay in the absence or presence of T151742. Upon addition of T151742, Tagg was quantified for ERO1α, ERO1β, and LSD-1. Quantification of the average of n=3 independent experiments based on western blot analysis for ERO1α, ERO1β, and LSD-1. The dashed line ( - - - ) line represents the cut-off point at 10% which was used to define our aggregation temperature (Tagg) of individual proteins. FIG. 6B shows a MTT viability assay comparing potency between ERO1α inhibitor EN-460, T151742, and T151742 derivative SR-F-114, revealing SR-F-114 is about two-fold less effective in vitro versus T151742 and EN-460 in the PC-9 non-small cell lung cancer cell line. Combined n=3 independent experiments performed in quadruplicates ***p<0.001 by one-way ANOVA. FIG. 6C shows a validation of ERO1α knockout in the PC-9 cell line utilizing CRISPR. FIG. 6D shows two CRISPR deleted ERO1α clones that were subjected to treatment of T151742 as well as the non-targeted control PC-9 cell line. Cellular viability was determined by MTT assay. n=3 independent experiments performed in quadruplicates. ****p<0.0001; by two-way ANOVA.

### Evaluating the Efficacy of T151742 in the PC-9 Lung Cancer Cell Line

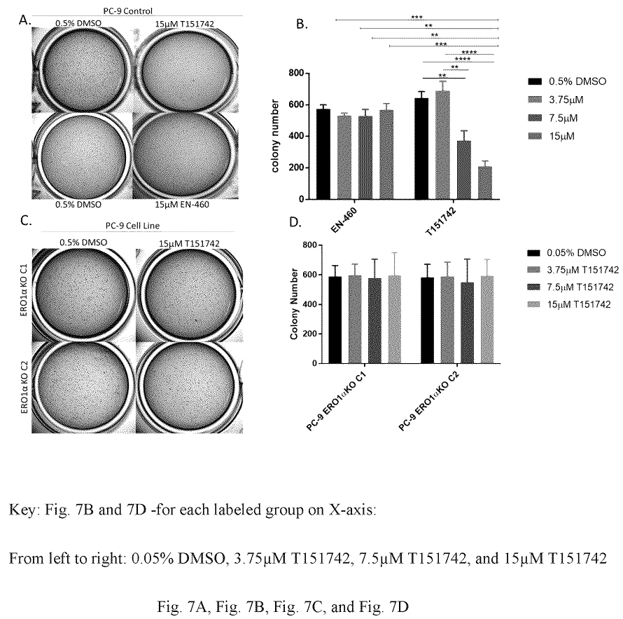

We utilized an MTT assay to determine the potency of the compounds for inhibiting cell growth. As shown in FIG. 6B, we compared T151742, EN-460, and SR-F-114. Consistent with the enzymatic assay T151742 was most the most potent while SR-F-114 was approximately two-fold less active. (T151742 IC₅₀=14.05±3.99 μM, EN-460 IC₅₀=16.62±4.34 μM, and SR-F-114 IC₅₀=32.36±6.01). We next asked whether T151742 induced inhibition of cell growth was dependent on ERO1α expression. As shown in FIG. 6C two ERO1α deleted clones in the PC-9 cell line were isolated. Using an MTT assay to determine overall cellar viability, a non-targeted control (scramble), and both ERO1α knockout clones was subjected to treatment with T151742 for 72 hours (See FIG. 6D). PC-9 ERO1α deleted cells were approximately 2-3-fold more resistant to T157742 treatment compared to the control cell line (PC-9 WT IC₅₀=14.13 μM±1.15, PC-9 ERO1α KO Clone 1 IC₅₀=32.23±1.09 μM and PC-9 ERO1α KO Clone 2 IC₅₀=41.54±1.08 μM. We were also able to determine that the IC₅₀ values in myeloma cells lines was 7-9 μM indicating that myeloma cells demonstrate increased sensitivity to inhibition of ERO1α. These data suggest ERO1α maybe important to further credential as a target for the treatment of multiple myeloma. Importantly, the normal lung epithelial cell line Beas-2B the IC₅₀ value was found to be >100 μM suggesting that cancer cells are more dependent on ERO1α compared to normal cells (see FIG. 8) for IC₅₀ values of all cell lines tested). Though only slight differences were observed between T151742 and EN-460 we tested the effects of these compounds in a clonogenic assay which is a better test for inhibition of self-renewal. Utilizing soft agar assays we were able to determine that T151742 was significantly more potent at reducing colony number in a concentration-dependent manner in comparison to EN-460 (FIG. 7A and FIG. 7B). PC-9 ERO1α knockout clones were found to be completely resistant to T151742 using a clonogenic assay as an endpoint (FIG. 7C and FIG. 7D). Together, these data suggest that the activity of T151742 is dependent upon ERO1α expression when using clonogenicity as an endpoint.

FIG. 7A, FIG. 7B, FIG. 7C, and FIG. 7D show soft agar clonogenic assays that demonstrate that T151742 is more potent compared to EN-460 and ERO1α knockout clones are resistant to T151742 treatment. FIG. 7A and FIG. 7B show 1000 cells/well were plated in a 12-well plate in triplicate with either 0.5% DMSO, T151742, or EN-460 and allowed to form colonies for 14 days. Colonies were subsequently stained with 0.01% Crystal Violet, imaged, and quantified using ImageJ. Fig. &C and FIG. 7D show 1500 cells/well were plated in a 12-well in triplicate with either 0.5% DMSO or T151742 and allowed to form colonies for 14 days. Colonies were stained with 0.01% Crystal Violet, imaged, and quantified using ImageJ.

### Conclusions:

In conclusion, we identified T151742 as the most potent aurone analog which demonstrated activity against ERO1α. With T151742 having activity against ERO1α in a recombinant assay system and binds the target using in vitro assays. Aurone and chalcone molecules synthesized showed similar activity, but no derivatives made were found to be more potent than T151742. We were also able to demonstrate that T151742 activity was completely dependent on ERO1α expression using soft agar clonogenic assay as an endpoint. Additionally, our data indicate that clonogenic assays may be a better endpoint compared to proliferation or a death assay when screening compounds against ERO1α. The CETSA assay indicates T151742 has specificity towards ERO1α over other flavoenzymes, and these data indicates this assay can be used to validate ERO1α target coverage using in vitro. Further studies are required to determine whether the CETSA assay can be used to determine target coverage of ERO1α using in vivo models of cancer. Lastly, we were able to determine through structure-activity relationship built upon here that both the hydroxyl group at position 5 on the A ring and the tertiary amine group on the B ring are important for activity against ERO1α, as all derivatives with changes at these positions lost efficacy towards ERO1α in our recombinant system. Ultimately, our results suggest the aurone scaffold can be exploited to further develop novel specific inhibitors to pharmacologically credential the target ERO1α using in vitro models of cancer. Future studies are warranted to determine whether T151742 can be used to credential ERO1α using in vivo models of cancer. Studies using a clonogenic assay comparing EN-460 and T151742 suggest that specificity maybe more important for targeting self-renewal or clonogenic growth in soft-agar. However, further studies will be required to determine whether a promiscuous inhibitor targeting multiple FAD containing enzymes will be more effective compared to specific targeting of ERO1α for the treatment of cancer. The experimental data presented demonstrates that covalent modifiers of FAD do not inhibit ERO1α enzymatic activity and T157142 does not inhibit LSD-1 enzymatic activity.

### Materials and Methods

Compounds used in the assays were obtained from commercial sources. EN-460 (Sigma Aldrich, CAT #328501), LSD-1 Inhibitor IV (Sigma Aldrich, CAT #489479), and Tranylcypromine (Sigma Aldrich, CAT #616431) were purchased. All T151742 derivatives that were used in experiments were made synthetically and experimental procedures on synthetic strategy are set forth herein.

Protein Expression, Purification, and Enzymatic assays: Human ERO1α (a.a. 22-468) containing hyperactivating triple mutation (C104A, C131A, and C166A¹⁹) was purified as previously described⁶. Human PDI (a.a. 18-479) was synthesized and purified as previously described⁶. Quantification of enzymatic activity of ERO1α was performed using the Amplex Red as previously described⁶.

Cell Lines: PC-9 cell line used was obtained from Sigma Aldrich and grown in RPMI 1640 medium with 10% fetal bovine serum and 1% penicillin/streptomycin. HCC4006, U266, MM1.s, and Beas-2B cell lines were obtained from ATCC and cultured in either RPMI 1640 or DMEM with 10% fetal bovine serum and 1% penicillin/streptomycin. All cell lines were also checked for mycoplasma every six months and subjected to short tandem repeat (STR) analysis for cell line validation.
CRISPR Cas9: CRISPR Guide RNA 2 targeting ERO1α and CRISPR Guide RNA 4 were purchased from Genscript in the pLentiCRISPR V2 plasmid containing puromycin/ampicillin selection marker. Lentivirus was made from HEK 293T cells by co-transfection with GuideRNA of interest, pVSVG, and pPAX2 plasmids. Lentivirus was collected after 48 hours and concentrated using 40% PEG8000. Concentrated virus was used to infect PC-9 cells. Cells were placed under puromycin selection after 72 hours and cloned out using limiting dilutions.
Lineweaver-Burke: Varying concentration of PDI (0 mg/mL-0.25 mg/mL) and T151742 were assayed using the Amplex red kit as described above. The reaction was incubated for 30 min at 37° C. for 30 minutes. Plate was then read on the BioTek Cytation 5 at 530/590 nm.
MTT: Cells were plated at 10,000 cells/well in quadruplets in a 96 well plate and allowed to attach for 24 hours. EN-460 or T151742 were added at increasing concentrations (100 μM, 50 μM, 25 μM, 12.5 μM, 6.25 μM, 3.125 μM. 1.5625 μM) with DMSO as control and allowed to incubate at 37° C. and 5% CO₂ for an additional 72 hours. After 72 hours MTT (3-(4,5-Dimethylthiazol-2-yl)-2,5-Diphenyltetrazolium Bromide) dye (2 mg/mL) was added to each well and incubated at 37° C. and 5% CO₂ for 4 hours. Dye and media were aspirated and DMSO was added to plate and placed on a rocker at room temperature for 10 min. Plate was then read at 570 nm. Treated wells were all compared to DMSO control wells to calculate percent growth inhibition and the IC₅₀ was calculated using GraphPad (log(inhibitor) vs growth inhibition).
Soft Agar Clonogenic: A base layer consisting of 1 mL 1% Agar in RPMI-1640 was set and allowed to cool to room temperature. A top layer was then added containing 500 μL 0.5% agar in RPMI-1640 and cells at indicated concentration on figure legend. Once agar had solidified at 37° C., 500 μL of RPMI-1640 culture media was added to the top. Plates were incubated for 14 days and were then stained with 0.01% Crystal Violet and imaged. Images were then quantified using ImageJ.
Cellular Thermal Shift Assay: PC-9 cell line (9 million cells) were incubated with either 0.5% DMSO control or T151742 at 100 μM for 1 hr at 37° C. Cells were aliquoted at 1 million cells per PCR tube in PBS and heated in Bio-Rad T100 thermal cycler at indicated temperatures (RT, 46° C., 48° C., 50° C., 52° C., 54° C., 56°, 58° C., and 60° C.) for 3 minutes and aggregation temperature was determined by western blot analysis. The CETSA Protocol was adapted from Jafari et al⁴⁵. ERO1α antibody (Santa Cruz Cat #sc-365526), ERO1β (Proteintech Cat #11261-2-AP), and LSD-1 (cell signaling Cat #21395). Secondary antibodies used for detection include anti-rabbit IgG (Jackson laboratories Cat #111-035-003) and anti-mouse IgG (Jackson laboratories Cat #115-035-003). Substrate used for western blot detection was ECL-A/ECL-B (Thermo Fisher CAT #32209). All western blot images and analysis was completed using the Amersham imager 680.
Biacore Analysis: Affinity analysis was carried out using a Biacore T200 instrument (GE Healthcare Life Sciences and analysis was performed at Creative Biolabs, Shirley, NY). Briefly, ERO1α protein was directly immobilized on the chip (Serie S-type CM5) using an amine coupling kit (GE Healthcare Life Sciences). The protein was diluted into was diluted into 50 μg/ml with the immobilization buffer. The CM5 sensor surface was activated using 400 mM EDC and 100 mM NHS, injected at a flow rate of 10 μl/min, with a contact time of 420 s. ERO1α (50 μg/ml) was injected into FC2 at a flow rate of 10 μl/min. The amount of ERO1α immobilized was about 10000 RU. Then, cholamine was injected for blocking at a flow rate of 10 μl/min, with a contact time of 420 s. T151742 was serially diluted with the running buffer to give a concentration of 100, 50, 25, 12.5, 6.25, 3.125, 1.563, 0 μM. Data analysis was performed on the Biacore T200 computer and with the Biacore T200 evaluation software.

### Experimental Procedures

Chemistry. Materials & Instruments

All reagents and solvents were obtained in highest grade possible from commercial sources and used as received, unless otherwise specified. Purification of the compounds from the reactions was performed by trituration or either by flash chromatography or HPLC. Reactions were monitored for progress by thin layer chromatography (TLC) on pre-coated glass plates (Merck KGaA Silicagel 60 F254). Flash chromatography was performed using silica gel (60 Å, 60-200 μm, Acros Organics). HPLC purification was performed on Agilent 1100 series preparatory system using Spirit Protein 300 C8 5 μm 25×2.12 column (Aapptec®) at 254 nm wavelength. HPLC analysis was performed on Agilent 1100 Series instrument using HyperClone 5μ C8 (MOS)1 150×2.00 mm 5μ micron column (Phenomenex®) at 254 nm wavelength. ¹H NMR, ¹³C NMR and ¹⁹F NMR spectra were recorded on a JEOL 400 MHz spectrometer. ¹H spectra were recorded at 400 MHz and ¹³C NMR spectra were recorded using a proton-decoupled pulse sequence run at 100 MHz. ¹⁹F spectra were recorded at 376 MHz. Chemical shifts (δ) are expressed in parts per million (ppm) and the shifts were corrected as δ 7.26 for CDCl₃, δ 2.54 for DMSO-d₆ and for proton. Chemical shifts were corrected as δ 77.36 for CDCl₃, δ 40.45 for DMSO-d₆ for carbon NMRs. Signal splitting patters were described as singlet (s), doublet (d), triplet (t), quartet (q), double doublet (dd) or a multiplet (m). Coupling constants (J) were quoted to the nearest 0.1 Hz. Due to trans/cis isomerization of some unhindered molecules; more signals were observed in ¹H and ¹³C spectra than would be expected for the pure trans product. High resolution mass measurements were carried out on Thermo Fisher Q Exactive Orbitrap mass spectrometer.

### Synthesis of ERO1α Inhibitors.

6-Hydroxy-2-benzylidene-1-benzofuran-3-one derivatives were synthesized following previously reported methods. Initially, compounds 114-127 were synthesized by Claisen-Schmidt condensation between 6-hydroxy-3-coumaranone and substituted benzaldehydes in good yields in the presence of aqueous sodium hydroxide.^{46,47} Compounds 128-131 were synthesized using similar conditions from acetophenones as shown in Scheme 1.^{48,49} Introduction of the hydrophilic and hydrophobic, electron withdrawing and electron donating substitutions at ortho-, meta-, and para-positions allowed to investigate their effects on the biological activity.

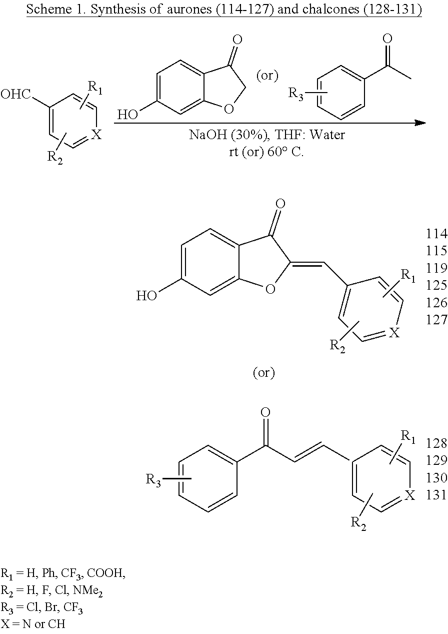

### General Method of Synthesis

To a mixture of 6-Hydroxy-3-coumaranone or substituted acetophenone, (1.4 mmol) in tetrahydrofuran and water (10 mL, typically 1:1 ratio), an equivalent amount of aryl aldehydes was added and stirred at room temperature. 30% sodium hydroxide (500 μL) was added dropwise to the above solution and the resulting reaction was heated to gentle reflux for 2-6 hours. Upon the completion of the reaction by thin layer chromatography, reaction mixture was neutralized by the dropwise addition of acetic acid. Solvents were removed under reduced pressure and the resulting crude was extracted into ethyl acetate (100 mL) with saturated brine washings (2×25 mL). The organic layer was dried over anhydrous sodium sulfate and concentrated to afford the crude product that was purified by trituration in ether. The product from the resulting supernatant and from reaction where precipitation did not form was subjected to either flash chromatography (10% ethyl acetate, 90% hexanes) or reverse phase HPLC to obtain (recover) final compounds as solids (yellow to orange red).

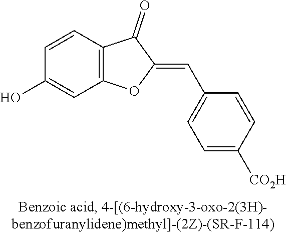 [SMILES: O=C(O)c1ccc(/C=C2\Oc3cc(O)ccc3C2=O)cc1]

¹H NMR (400 MHz, DMSO-d₆) δ 11.36-11.33 (s, 1H), 8.12-8.04 (q, J₁=14, J₂=8.4, 4H), 7.72-7.67 (d, J=8.4, 1H), 6.90-6.88 (s, 1H), 6.87-8.85 (d, J=2.4, 1H), 6.80-6.72 (dd, J₂=2.4, J₁=8.8, 1H); ¹³C NMR (100 MHz, DMSO-d₆) δ 182.35, 169.07, 167.78, 167.75, 149.24, 137.24, 131.92, 131.88, 130.65, 127.13, 114.21, 113.45, 109.8, 99.68; ¹³C DEPT NMR (100 MHz, DMSO-d₆) δ 131.63, 130.40, 126.89, 113.96, 109.55, 99.43; HRMS (ESI-TOF) m/z [M−H] negative calc'd. for C₁₆H₉O₅ 281.0455, found 281.0456, yield 63%, yellow solid. Melting Point=317-326.5° C.

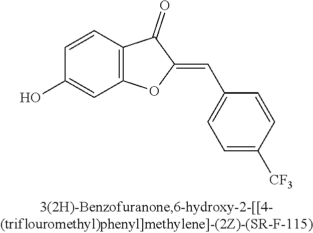 [SMILES: O=C1/C(=C/c2ccc(C(F)(F)F)cc2)Oc2cc(O)ccc21]

¹H NMR (400 MHz, DMSO-d₆) δ 11.41-11.31 (s, 1H), 8.22-8.15 (d, J=8.0, 2H), 7.92-7.85 (d, J=8.0, 2H), 7.72-7.67 (d, J=8.4, 1H), 6.94-6.90 (s, 1H), 6.86-6.84 (d, J=2.0, 1H), 6.80-6.76 (dd, J₂=2.4, J₁=8.8, 1H); ¹³C NMR (100 MHz, DMSO-d₆) δ 184.20, 170.46, 168.96, 150.46, 137.62, 132.54, 127.30, 126.72, 126.68, 114.47, 114.23, 110.19, 99.68; ¹³C DEPT NMR (100 MHz, DMSO-d₆) δ 132.09, 126.95, 126.42, 114.02, 108.94, 99.40; HRMS (ESI-TOF) m/z [M−H] negative calc'd. for C₁₆H₈F₃O₃ 305.0431, found 305.0432. 76% yield, light yellow solid. Melting Point=241.9° C.

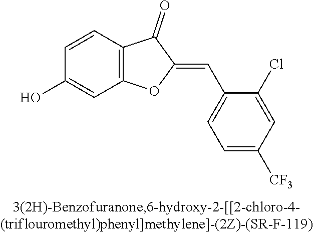 [SMILES: O=C1/C(=C/c2ccc(C(F)(F)F)cc2Cl)Oc2cc(O)ccc21]

¹H NMR (100 MHz, DMSO-d₆) δ 8.53-8.47 (s, 1H), 7.85-7.79 (d, J=8.0, 1H), 7.74-7.68 (dd, J₁=8.4, J₂=2.0, 1H), 7.26-7.20 (d, J=8.8, 1H), 6.64-6.60 (s, 1H), 6.06-6.00 (dd, J₂=1.6, J₁=8.8, 1H), 5.83 (d, J=1.2, 1H); ¹³C NMR (100 MHz, DMSO-d₆) δ 182.47, 175.91, 170.49, 154.07, 133.29, 131.82, 129.31, 128.99, 127.65, 127.61, 126.33, 126.30, 125.92, 103.96, 99.62, 97.92; ¹³C DEPT NMR (100 MHz, DMSO-d₆) δ 131.89, 127.64, 126.33, 125.91, 121.09, 99.61, 97.92; HRMS (ESI-TOF) m/z [M−H] negative calc'd. for C₁₆H₇ClF₃O₃ 339.0041, found 339.0041. 85% yield, orange solid. Melting Point=310.5° C.

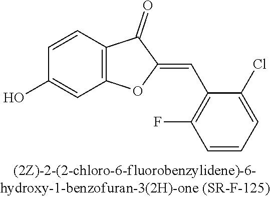 [SMILES: O=C1/C(=C/c2c(F)cccc2Cl)Oc2cc(O)ccc21]

¹H NMR (100 MHz, DMSO-d₆) δ 11.43-11.35 (s, 1H), 7.73-7.67 (d, J=8.0, 1H), 7.62-7.48 (m, 2H), 7.46-7.38 (t, J=8.4, 1H), 6.80-6.68 (m, 3H); ¹³C NMR (100 MHz, DMSO-d₆) δ 182.33, 168.86, 167.54, 148.41, 141.98, 140.12, 132.63, 132.18, 130.00, 128.96, 128.08, 127.68, 126.93, 114.05, 113.75, 110.98, 99.61; ¹³C DEPT NMR (100 MHz, DMSO-d₆) δ 132.39, 127.17, 126.30, 115.81, 113.99, 101.28, 99.25; HRMS (ESI-TOF) m/z [M−H] negative calc'd. for C₁₅H₇ClFO₃ 289.0073, found 289.0077. 89% yield, yellow solid. Melting Point=277.6° C.

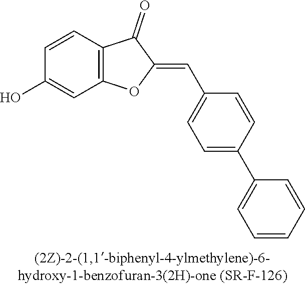 [SMILES: O=C1/C(=C/c2ccc(-c3ccccc3)cc2)Oc2cc(O)ccc21]

¹H NMR (400 MHz, DMSO-d₆) δ 11.31-11.26 (s, 1H), 8.12-8.06 (d, J=8.0, 2H), 7.88-7.83 (d, J=8.4, 2H), 7.82-7.76 (d, J=7.6, 2H), 7.71-7.66 (d, J=8.4, 1H), 7.58-7.50 (t, J=7.6, 2H), 7.48-7.41 (t, J=7.2, 1H), 6.91-6.85 (m, 2H), 6.8-6.75 (dd, J₂=2.0, J₁=8.8, 1H); ¹³C NMR (100 MHz, DMSO-d₆) δ 182.33, 168.86, 167.54, 148.42, 141.97, 140.13, 132.63, 132.18, 130.00, 128.95, 128.08, 127.68, 126.97, 114.05, 113.74, 110.97, 99.61; ¹³C DEPT NMR (100 MHz, DMSO-d₆) δ 132.36, 129.73, 128.69, 127.81, 127.41, 126.70, 113.78, 110.70, 99.34; HRMS (ESI-TOF) m/z [M−H] negative calc'd. for C₂₁H₁₃O₃ 313.0870, found 313.0872. 54% yield, yellow solid. Melting Point=275.3° C.

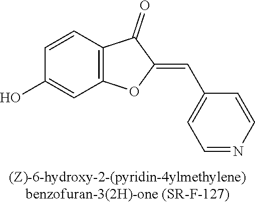 [SMILES: O=C1/C(=C/c2ccncc2)Oc2cc(O)ccc21]

¹H NMR (400 MHz, DMSO-d₆) δ 11.50-11.35 (bs, 1H), 8.76-8.68 (d, J=6.0, 2H), 7.91-7.85 (d, J=6.0, 2H), 7.73-7.67 (d, J=8.4, 1H), 6.88-6.85 (d, J=2.0, 1H), 6.82-6.80 (s, 1H), 6.80-6.76 (dd, J₁=8.4, J₂=2.0, 1H); ¹³C NMR (100 MHz, DMSO-d₆) δ 182.24, 169.24, 168.13, 151.25, 150.72, 140.18, 127.29, 125.32, 114.39, 113.18, 107.97, 99.77; ¹³C DEPT NMR (100 MHz, DMSO-d₆) δ 151.00, 127.04, 125.07, 114.14, 107.72, 99.52; HRMS (ESI-TOF) m/z [M−H] negative calc'd. for C₁₄H₈NO₃ 238.0510, found 238.0510. 67% yield, light yellow solid. Melting Point=289.5° C.

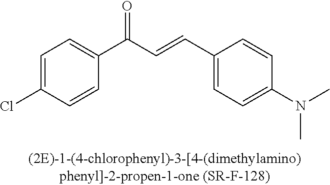 [SMILES: CN(C)c1ccc(/C=C/C(=O)c2ccc(Cl)cc2)cc1]

¹H NMR (400 MHz, DMSO-d₆) δ 8.20-8.14 (d, J=8.8, 2H), 7.78-7.61 (m, 6H), 6.81-6.76 (d, J=8.8, 2H), 3.07-3.03 (s, 6H); ¹³C NMR (100 MHz, DMSO-d₆) δ 188.36, 153.03, 146.65, 138.31, 137.93, 131.87, 131.04, 129.64, 122.79, 116.56, 112.64, 40.60; ¹³C DEPT NMR (100 MHz, DMSO-d₆) δ 146.65, 131.87, 131.03, 129.64, 116.56, 112.64, 40.60; HRMS (ESI-TOF) m/z [M+H] positive calc'd. for C₁₇H₁₇ClNO 286.0993, found 286.0986. 64% yield, orange solid. Melting Point=140.5° C.

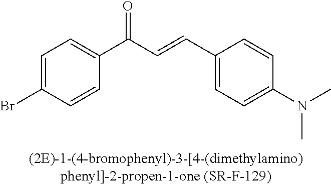 [SMILES: CN(C)c1ccc(/C=C/C(=O)c2ccc(Br)cc2)cc1]

¹H NMR (400 MHz, DMSO-d₆) δ 8.12-8.05 (d, J=8.4, 2H), 7.82-7.61 (m, 6H), 6.82-6.75 (d, J=8.8, 2H), 3.05 (s, 6H); ¹³C NMR (400 MHz, DMSO-d₆) δ 188.55, 153.03, 146.67, 138.26, 132.58, 131.86, 131.16, 127.43, 122.78, 116.53, 112.64, 40.61; ¹³C DEPT NMR (100 MHz, DMSO-d₆) δ 146.68, 132.59, 131.88, 131.17, 116.53, 112.63, 40.60; HRMS (ESI-TOF) m/z [M+H] positive calc'd. for C₁₇H₁₇BrNO 330.0488, found 330.0478. 71% yield, orange solid. Melting Point=289.5° C. Melting Point=143.6° C.

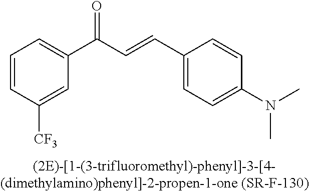 [SMILES: CN(C)c1ccc(/C=C/C(=O)c2cccc(C(F)(F)F)c2)cc1]

¹H NMR (400 MHz, DMSO-d₆) δ 8.50-8.43 (d, J=7.6, 1H), 8.42-8.38 (s, 1H), 8.07-8.00 (d, J=8.0, 1H), 7.88-7.74 (m, 5H), 6.84-6.76 (d, J=9.2, 2H), 3.22-3.02 (s, 6H); ¹³C NMR (100 MHz, DMSO-d₆) δ 188.18, 153.13, 147.32, 140.02, 133.08, 132.10, 130.86, 130.59, 130.28, 129.72 (d, J=3.8), 126.27, 125.43 (d, J=3.8), 123.56, 122.73, 116.29, 112.59, 40.59; ¹³C DEPT NMR (100 MHz, DMSO-d₆) δ 147.32, 133.08, 132.10, 130.86, 129.72 (d, J=3.8), 125.45, 116.29, 112.59, 40.59; HRMS (ESI-TOF) m/z [M+H] positive calc'd. for C₁₈H₁₇F₃NO 320.1257, found 320.1248.58% yield, orange yellow solid. Melting Point=83.3° C.

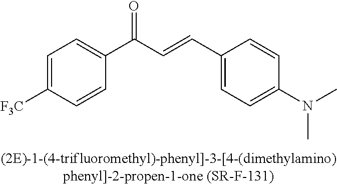 [SMILES: CN(C)c1ccc(/C=C/C(=O)c2ccc(C(F)(F)F)cc2)cc1]

¹H NMR (400 MHz, CDCl₃) δ 8.20-8.05 (d, J=8.4, 2H), 7.94-7.68 (m, 3H), 7.64-7.45 (d, J=8.8, 2H), 7.35-7.16 (d, J=15.2, 1H), 6.85-6.58 (d, J=8.4, 2H), 3.12-3.01 (s, 6H); ¹³C NMR (100 MHz, CDCl₃) δ 190.02, 152.67, 147.47, 142.45, 133.40 (q), 131.05, 128.90, 125.83, 125.52, 122.66, 116.67, 112.18, 40.44; ¹³C DEPT NMR (100 MHz, CDCl₃) δ 147.47, 131.04, 128.89, 125.83, 116.66, 112.16, 40.44; HRMS (ESI-TOF) m/z [M+H] positive calc'd. for C₁₈H₁₇F₃NO 320.1257, found 320.1247. 86% yield, orange yellow solid. Melting Point=142.1° C.

FIG. 8 shows that inhibitors that covalently modify FAD do not inhibit Ero1α activity. The MOA inhibitor Tranylcypromine and LSD-1 inhibitor IV mechanistically inhibit LSD-1 via covalent modification of FAD and were used to generate a concentration response. Experiments were performed in triplicates and shown is the mean and standard deviation of three independent experiments.

FIG. 9 shows that LSD-1 retains demethylase activity against dimethylated lysine N-terminal tail of histone 3 (H3) upon treatment with T151742. T151742 was used to generate a concentration response. As shown, T151742 did not inhibit LSD-1 demethylase activity at concentrations ≤25 μM using the LSD-1 inhibitor screening assay kit (Cayman chemical Item No: 700120). Experiments performed in triplicates and shown is the mean and standard deviation of three independent experiments.

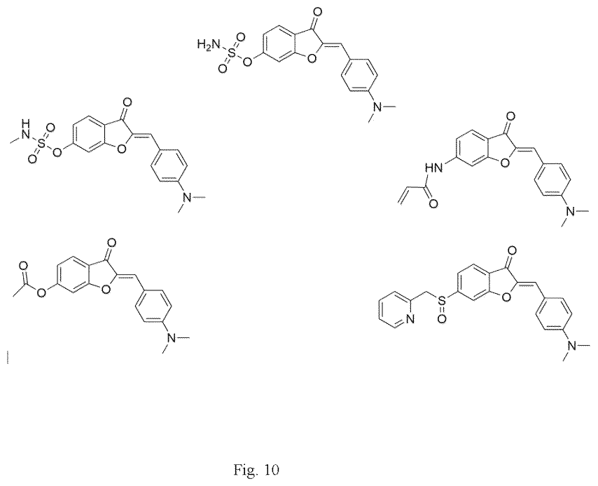

FIG. 10 shows structures of compounds of the present invention.

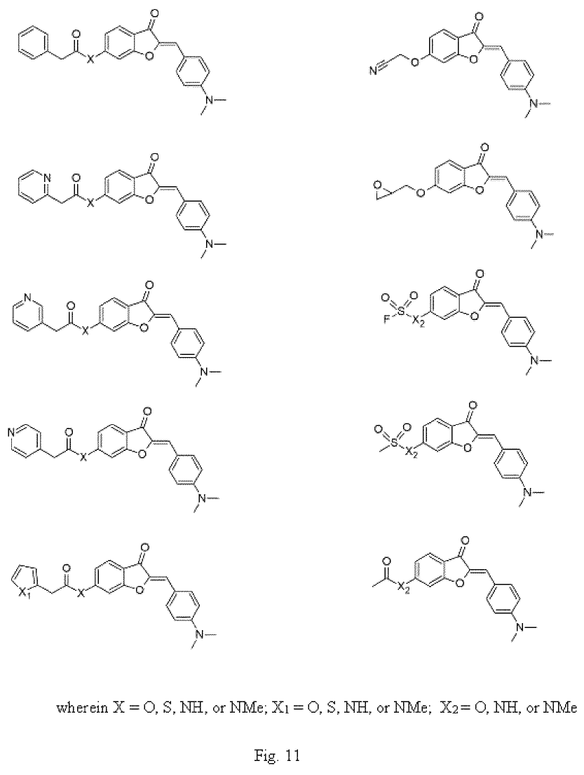

FIG. 11 shows structures of compounds of the present invention.

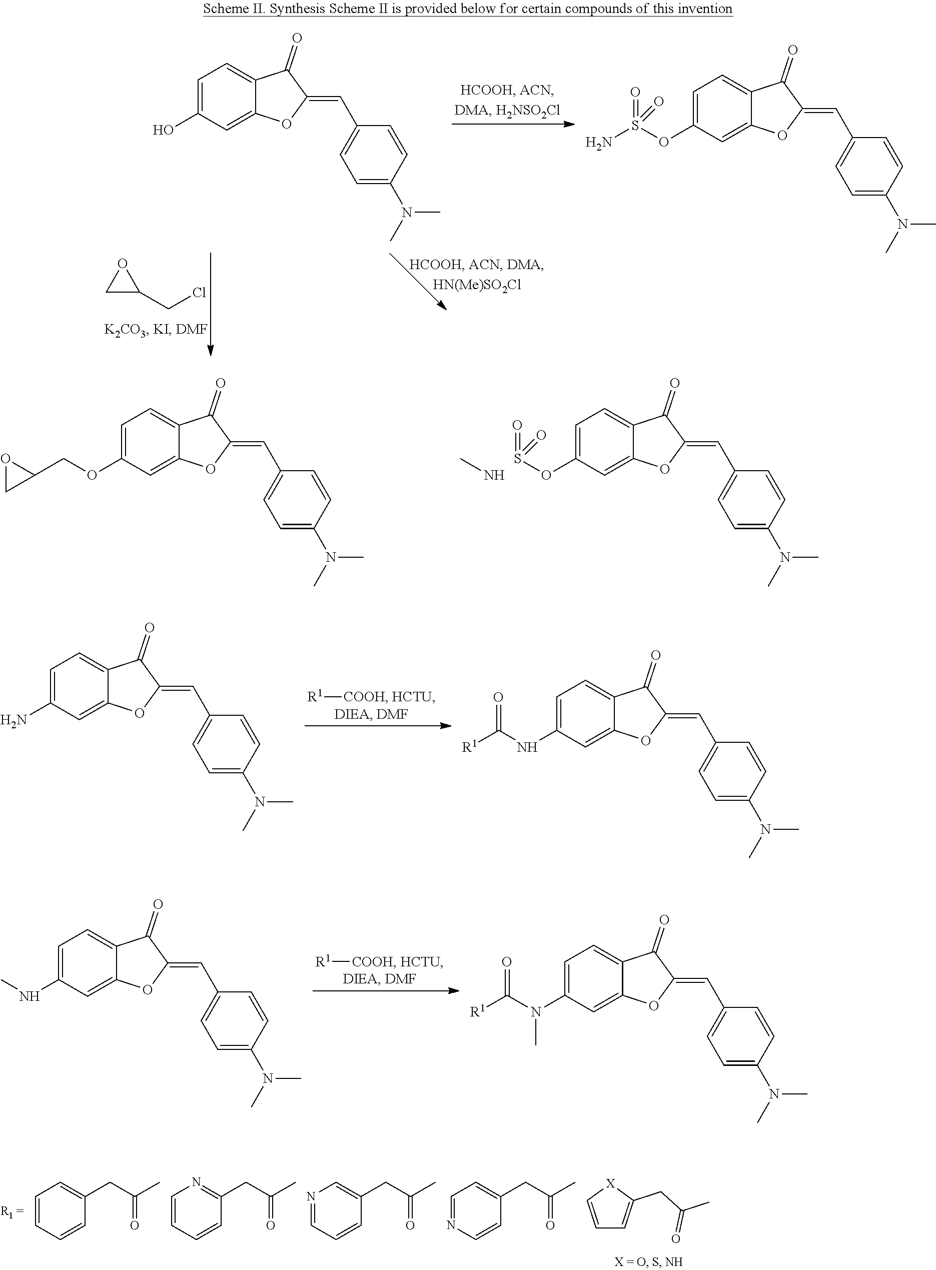

Table 2 shows IC50 values for T151742 were determined by MTT assay using a non-linear regression. Cancer cell lines were more sensitive to treatment with T151752 versus normal lung epithelium. IC50's reported are averages of three independent experiments and were performed in quadruplicates.

TABLE 2

| Cell Line | IC₅₀ Avg ± s.d. (n = 3) | Cell type |
|---|---|---|
| PC-9 | 14.05 ± 3.99 μM | NSCLC (adenocarcinoma) |
| HCC4006 | 18.51 ± 14.66 μM | NSCLC (adenocarcinoma) |
| U266 | 8.93 ± 1.13 μM | Multiple Myeloma |
| MM1.s | 7.45 ± 1.12 μM | Multiple Myeoloma |
| Beas-2B | >100 μM | Normal Lung Epithelium |

### REFERENCES

1. Herbst, R. S.; Morgensztern, D.; Boshoff, C., The biology and management of non-small cell lung cancer. Nature 2018, 553 (7689), 446-454.
2. Abbosh, C.; Birkbak, N. J.; Swanton, C., Early stage NSCLC—challenges to implementing ctDNA-based screening and MRD detection. Nat Rev Clin Oncol 2018, 15 (9), 577-586.
3. Chaudhuri, A. A.; Chabon, J. J.; Lovejoy, A. F.; Newman, A. M.; Stehr, H.; Azad, T. D.; Khodadoust, M. S.; Esfahani, M. S.; Liu, C. L.; Zhou, L.; Scherer, F.; Kurtz, D. M.; Say, C.; Carter, J. N.; Merriott, D. J.; Dudley, J. C.; Binkley, M. S.; Modlin, L.; Padda, S. K.; Gensheimer, M. F.; West, R. B.; Shrager, J. B.; Neal, J. W.; Wakelee, H. A.; Loo, B. W., Jr.; Alizadeh, A. A.; Diehn, M., Early Detection of Molecular Residual Disease in Localized Lung Cancer by Circulating Tumor DNA Profiling. Cancer Discov 2017, 7 (12), 1394-1403.
4. Nair, R. R.; Tolentino, J.; Hazlehurst, L. A., The bone marrow microenvironment as a sanctuary for minimal residual disease in CML. Biochem Pharmacol 2010, 80 (5), 602-12.
5. Ootsuka, S.; Asami, S.; Sasaki, T.; Yoshida, Y.; Nemoto, N.; Shichino, H.; Chin, M.; Mugishima, H.; Suzuki, T., Useful markers for detecting minimal residual disease in cases of neuroblastoma. Biol Pharm Bull 2008, 31 (6), 1071-4.
6. Hayes, K. E.; Batsomboon, P.; Chen, W. C.; Johnson, B. D.; Becker, A.; Eschrich, S.; Yang, Y.; Robart, A. R.; Dudley, G. B.; Geldenhuys, W. J.; Hazlehurst, L. A., Inhibition of the FAD containing ER oxidoreductin 1 (Ero1) protein by EN-460 as a strategy for treatment of multiple myeloma. Bioorg Med Chem 2019, 27 (8), 1479-1488.
7. Tanaka, T.; Kajiwara, T.; Torigoe, T.; Okamoto, Y.; Sato, N.; Tamura, Y., Cancer-associated oxidoreductase ERO1-alpha drives the production of tumor-promoting myeloid-derived suppressor cells via oxidative protein folding. J Immunol 2015, 194 (4), 2004-10.
8. Tanaka, T.; Kutomi, G.; Kajiwara, T.; Kukita, K.; Kochin, V.; Kanaseki, T.; Tsukahara, T.; Hirohashi, Y.; Torigoe, T.; Okamoto, Y.; Hirata, K.; Sato, N.; Tamura, Y., Cancer-associated oxidoreductase ERO1-alpha drives the production of VEGF via oxidative protein folding and regulating the mRNA level. Br J Cancer 2016, 114 (11), 1227-34.
9. Tanaka, T.; Kutomi, G.; Kajiwara, T.; Kukita, K.; Kochin, V.; Kanaseki, T.; Tsukahara, T.; Hirohashi, Y.; Torigoe, T.; Okamoto, Y.; Hirata, K.; Sato, N.; Tamura, Y., Cancer-associated oxidoreductase ERO1-alpha promotes immune escape through up-regulation of PD-L1 in human breast cancer. Oncotarget 2017, 8 (15), 24706-24718.
10. Wang, Y.; Zhang, L.; He, Z.; Deng, J.; Zhang, Z.; Liu, L.; Ye, W.; Liu, S., Tunicamycin induces ER stress and inhibits tumorigenesis of head and neck cancer cells by inhibiting N-glycosylation. Am J Transl Res 2020, 12 (2), 541-550.
11. Yang, S.; Yang, C.; Yu, F.; Ding, W.; Hu, Y.; Cheng, F.; Zhang, F.; Guan, B.; Wang, X.; Lu, L.; Rao, J., Endoplasmic reticulum resident oxidase ERO1-Lalpha promotes hepatocellular carcinoma metastasis and angiogenesis through the S1PR1/STAT3/VEGF-A pathway. Cell Death Dis 2018, 9 (11), 1105.
12. Neims, A. H.; Hellerman, L., Flavoenzyme catalysis. Annu Rev Biochem 1970, 39, 867-88.
13. Cabibbo, A.; Pagani, M.; Fabbri, M.; Rocchi, M.; Farmery, M. R.; Bulleid, N. J.; Sitia, R., ERO1-L, a human protein that favors disulfide bond formation in the endoplasmic reticulum. J Biol Chem 2000, 275 (7), 4827-33.
14. Fagioli, C.; Mezghrani, A.; Sitia, R., Reduction of interchain disulfide bonds precedes the dislocation of Ig-mu chains from the endoplasmic reticulum to the cytosol for proteasomal degradation. J Biol Chem 2001, 276 (44), 40962-7.
15. Frand, A. R.; Kaiser, C. A., The ERO1 gene of yeast is required for oxidation of protein dithiols in the endoplasmic reticulum. Mol Cell 1998, 1 (2), 161-70.
16. Mezghrani, A.; Fassio, A.; Benham, A.; Simmen, T.; Braakman, I.; Sitia, R., Manipulation of oxidative protein folding and PDI redox state in mammalian cells. EMBO J 2001, 20 (22), 6288-96.
17. Tu, B. P.; Weissman, J. S., Oxidative protein folding in eukaryotes: mechanisms and consequences. J Cell Biol 2004, 164 (3), 341-6.
18. Tu, B. P.; Weissman, J. S., The FAD- and O(2)-dependent reaction cycle of Ero1-mediated oxidative protein folding in the endoplasmic reticulum. Mol Cell 2002, 10 (5), 983-94.
19. Inaba, K.; Masui, S.; Iida, H.; Vavassori, S.; Sitia, R.; Suzuki, M., Crystal structures of human Ero1alpha reveal the mechanisms of regulated and targeted oxidation of PDI. EMBO J 2010, 29 (19), 3330-43.
20. Ron, D.; Walter, P., Signal integration in the endoplasmic reticulum unfolded protein response. Nat Rev Mol Cell Biol 2007, 8 (7), 519-29.
21. Sevier, C. S.; Kaiser, C. A., Ero1 and redox homeostasis in the endoplasmic reticulum. Biochim Biophys Acta 2008, 1783 (4), 549-56.
22. Zhang, J.; Zhu, Q.; Wang, X.; Yu, J.; Chen, X.; Wang, J.; Wang, X.; Xiao, J.; Wang, C. C.; Wang, L., Secretory kinase Fam20C tunes endoplasmic reticulum redox state via phosphorylation of Ero1alpha. EMBO J 2018, 37 (14).
23. Johnson, B. D.; Geldenhuys, W. J.; Hazlehurst, L. A., The Role of ERO1alpha in Modulating Cancer Progression and Immune Escape. J Cancer Immunol (Wilmington) 2020, 2 (3), 103-115.
24. Hanahan, D.; Weinberg, R. A., Hallmarks of cancer: the next generation. Cell 2011, 144 (5), 646-74.
25. Majmundar, A. J.; Wong, W. J.; Simon, M. C., Hypoxia-inducible factors and the response to hypoxic stress. Mol Cell 2010, 40 (2), 294-309.
26. Li, X.; Zhang, K.; Li, Z., Unfolded protein response in cancer: the physician's perspective. J Hematol Oncol 2011, 4, 8.
27. Sano, R.; Reed, J. C., ER stress-induced cell death mechanisms. Biochim Biophys Acta 2013, 1833 (12), 3460-3470.
28. Yadav, U. C.; Rani, V.; Deep, G.; Singh, R. K.; Palle, K., Oxidative Stress in Metabolic Disorders: Pathogenesis, Prevention, and Therapeutics. Oxid Med Cell Longev 2016, 2016, 9137629.
29. Neelamegam, R.; Ricq, E. L.; Malvaez, M.; Patnaik, D.; Norton, S.; Carlin, S. M.; Hill, I. T.; Wood, M. A.; Haggarty, S. J.; Hooker, J. M., Brain-penetrant LSD1 inhibitors can block memory consolidation. ACS Chem Neurosci 2012, 3 (2), 120-128.
30. Blais, J. D.; Chin, K. T.; Zito, E.; Zhang, Y.; Heldman, N.; Harding, H. P.; Fass, D.; Thorpe, C.; Ron, D., A small molecule inhibitor of endoplasmic reticulum oxidation 1 (ERO1) with selectively reversible thiol reactivity. J Biol Chem 2010, 285 (27), 20993-1003.
31. Lee, J. C.; Lim, K. T.; Jang, Y. S., Identification of Rhus verniciflua Stokes compounds that exhibit free radical scavenging and anti-apoptotic properties. Biochim Biophys Acta 2002, 1570 (3), 181-91.
32. Bohm, B. A., Chalcones, Aurones and Dihydrochalcones. In The Flavonoids, Harborne, J. B.; Mabry, T. J.; Mabry, H., Eds. Springer US: Boston, MA, 1975; pp 442-504.
33. Panche, A. N.; Diwan, A. D.; Chandra, S. R., Flavonoids: an overview. J Nutr Sci 2016, 5, e47.
34. Brown, J. P., A review of the genetic effects of naturally occurring flavonoids, anthraquinones and related compounds. Mutat Res 1980, 75 (3), 243-77.
35. Havsteen, B., Flavonoids, a class of natural products of high pharmacological potency. Biochem Pharmacol 1983, 32 (7), 1141-8.
36. Xu, H. X.; Lee, S. F., Activity of plant flavonoids against antibiotic-resistant bacteria. Phytother Res 2001, 15 (1), 39-43.
37. Geldenhuys, W. J.; Funk, M. O.; Van der Schyf, C. J.; Carroll, R. T., A scaffold hopping approach to identify novel monoamine oxidase B inhibitors. Bioorg Med Chem Lett 2012, 22 (3), 1380-3.
38. Barth, J.; Abou-El-Ardat, K.; Dalic, D.; Kurrle, N.; Maier, A. M.; Mohr, S.; Schutte, J.; Vassen, L.; Greve, G.; Schulz-Fincke, J.; Schmitt, M.; Tosic, M.; Metzger, E.; Bug, G.; Khandanpour, C.; Wagner, S. A.; Lubbert, M.; Jung, M.; Serve, H.; Schule, R.; Berg, T., LSD1 inhibition by tranylcypromine derivatives interferes with GFI1-mediated repression of PU.1 target genes and induces differentiation in AML. Leukemia 2019, 33 (6), 1411-1426.
39. Di Monte, D. A.; DeLanney, L. E.; Irwin, I.; Royland, J. E.; Chan, P.; Jakowec, M. W.; Langston, J. W., Monoamine oxidase-dependent metabolism of dopamine in the striatum and substantia nigra of L-DOPA-treated monkeys. Brain Res 1996, 738 (1), 53-9.
40. Gaweska, H.; Fitzpatrick, P. F., Structures and Mechanism of the Monoamine Oxidase Family. Biomol Concepts 2011, 2 (5), 365-377.
41. Heijnen, W. T.; De Fruyt, J.; Wierdsma, A. I.; Sienaert, P.; Birkenhager, T. K., Efficacy of Tranylcypromine in Bipolar Depression: A Systematic Review. J Clin Psychopharmacol 2015, 35 (6), 700-5.
42. Jia, Y.; Yun, C. H.; Park, E.; Ercan, D.; Manuia, M.; Juarez, J.; Xu, C.; Rhee, K.; Chen, T.; Zhang, H.; Palakurthi, S.; Jang, J.; Lelais, G.; DiDonato, M.; Bursulaya, B.; Michellys, P. Y.; Epple, R.; Marsilje, T. H.; McNeill, M.; Lu, W.; Harris, J.; Bender, S.; Wong, K. K.; Janne, P. A.; Eck, M. J., Overcoming EGFR(T790M) and EGFR(C797S) resistance with mutant-selective allosteric inhibitors. Nature 2016, 534 (7605), 129-32.
43. Kashima, K.; Kawauchi, H.; Tanimura, H.; Tachibana, Y.; Chiba, T.; Torizawa, T.; Sakamoto, H., CH7233163 Overcomes Osimertinib-Resistant EGFR-Del19/T790M/C797S Mutation. Mol Cancer Ther 2020, 19 (11), 2288-2297.
44. Kopecky, V., Jr.; Ettrich, R.; Pazderka, T.; Hofbauerova, K.; Reha, D.; Baumruk, V., Influence of ligand binding on structure and thermostability of human alpha1-acid glycoprotein. J Mol Recognit 2016, 29 (2), 70-9.
45. Jafari, R.; Almqvist, H.; Axelsson, H.; Ignatushchenko, M.; Lundback, T.; Nordlund, P.; Martinez Molina, D., The cellular thermal shift assay for evaluating drug target interactions in cells. Nat Protoc 2014, 9 (9), 2100-22.
46. Laurent Lunven, Hugues Bonnet, Samir Yahiaoui, Wei Yi, Laurene Da Costa, Marine Peuchmaur, Ah\grave{c}ene Boumendjel, and Sabine Chierici. Disruption of Fibers from the TauModel AcPHF6 by Naturally Occurring Aurones and Synthetic Analogues. ACS Chemical Neuroscience 2016, 7 (7), 995-1003; DOI: 10.1021/acschemneuro.6b00102
47. Sabrina Okombi, Delphine Rival, Sébastien Bonnet, Anne-Marie Mariotte, Eric Perrier, and Ahcène Boumendjel. Discovery of Benzylidenebenzofuran-3(2H)-one (Aurones) as Inhibitors of Tyrosinase Derived from Human Melanocytes. J. Med. Chem. 2006, 49, 329-333. DOI: 10.1021/jm050715i
48. Suvitha Shyam, Siddig Ibrahim Abdelwahab, Mohammed Ali-Al Mamary, and Syam Mohan. Synthesis of chalcones with anticancer activities. Molecules 2012, 17, 6179-6195; doi:10.3390/molecules17066179
49. Chunlin Zhuang, Wen Zhang, Chunquan Sheng, Wannian Zhang, Chengguo Xing, and Zhenyuan Miao. Chalcone: A privileged structure in medicinal chemistry. Chemical Reviews 2017 117 (12), 7762-7810. DOI: 10.1021/acs.chemrev.7b00020

It will be appreciated by those persons of ordinary skill in the art that changes could be made to the embodiments described above without departing from the broad inventive concept thereof. It is understood, therefore, that this invention is not limited to the particular embodiments disclosed, but it is intended to cover modifications that are within the spirit and scope of the invention, as defined by the appended claims.

1. A compound that is a derivative of sulfuretin that is an inhibitor of the endoplasmic reticulum oxidoreductin-1 alpha.

2. The compound of claim 1 selected from one of the group consisting of:

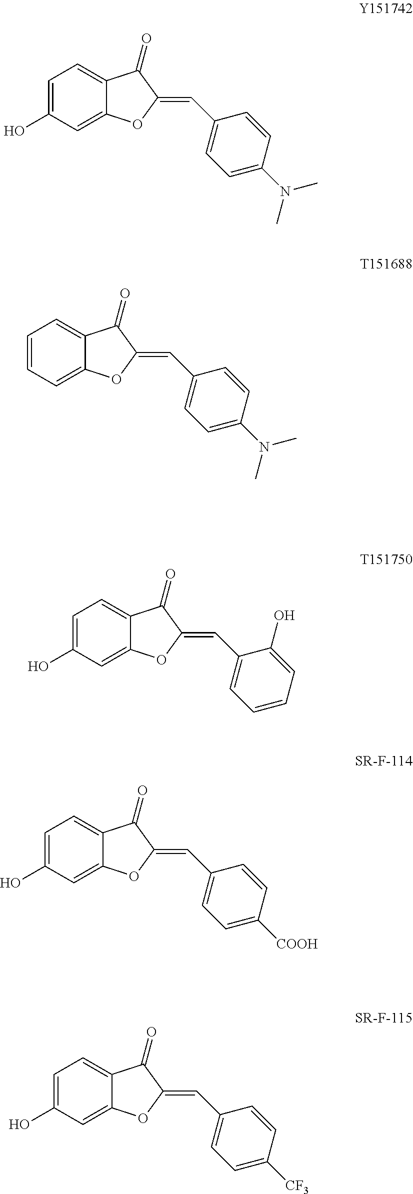 [SMILES: CN(C)c1ccc(/C=C2\Oc3cc(O)ccc3C2=O)cc1 ; CN(C)c1ccc(/C=C2\Oc3ccccc3C2=O)cc1 ; O=C1/C(=C/c2ccccc2O)Oc2cc(O)ccc21 ; O=C(O)c1ccc(/C=C2\Oc3cc(O)ccc3C2=O)cc1 ; O=C1/C(=C/c2ccc(C(F)(F)F)cc2)Oc2cc(O)ccc21]

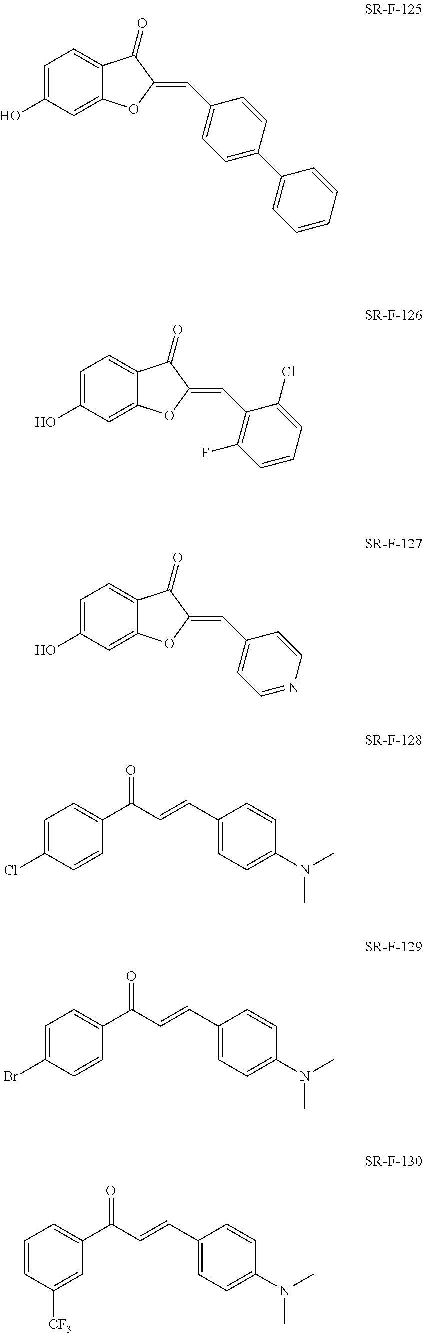 [SMILES: O=C1/C(=C/c2ccc(-c3ccccc3)cc2)Oc2cc(O)ccc21 ; O=C1/C(=C/c2c(F)cccc2Cl)Oc2cc(O)ccc21 ; O=C1/C(=C/c2ccncc2)Oc2cc(O)ccc21 ; CN(C)c1ccc(/C=C/C(=O)c2ccc(Cl)cc2)cc1 ; CN(C)c1ccc(/C=C/C(=O)c2ccc(Br)cc2)cc1 ; CN(C)c1ccc(/C=C/C(=O)c2cccc(C(F)(F)F)c2)cc1]

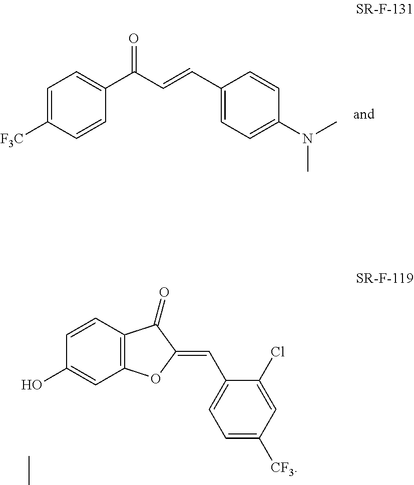 [SMILES: CN(C)c1ccc(/C=C/C(=O)c2ccc(C(F)(F)F)cc2)cc1 ; O=C1/C(=C/c2ccc(C(F)(F)F)cc2Cl)Oc2cc(O)ccc21]

3. The compound of claim 1 selected from one of the group consisting of: 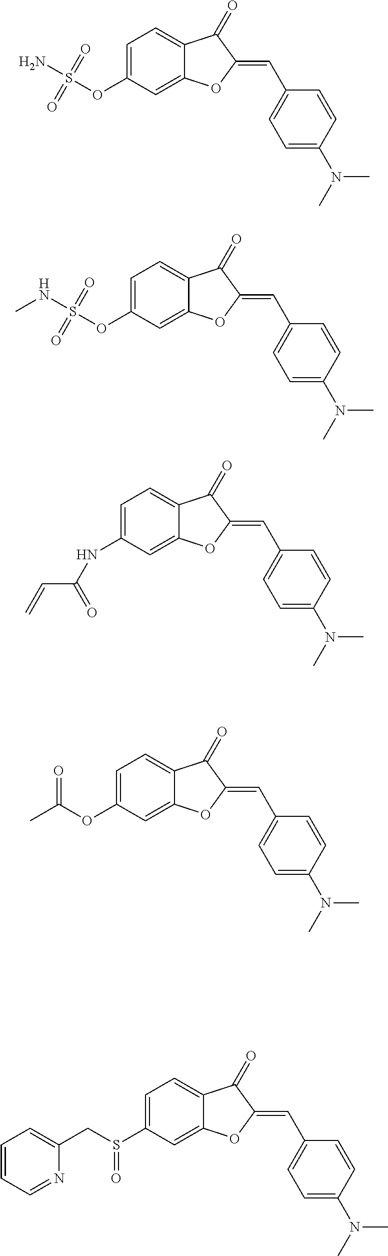 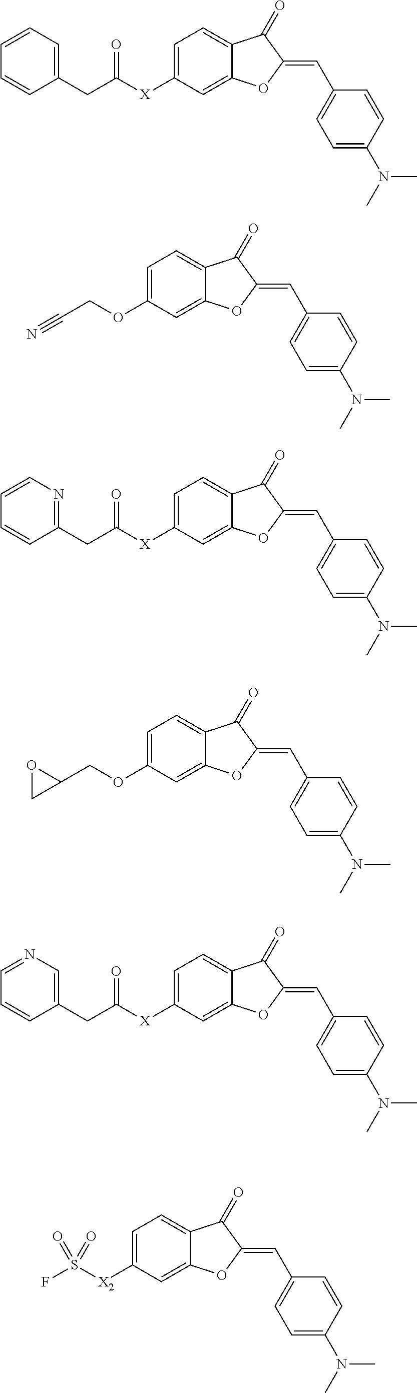 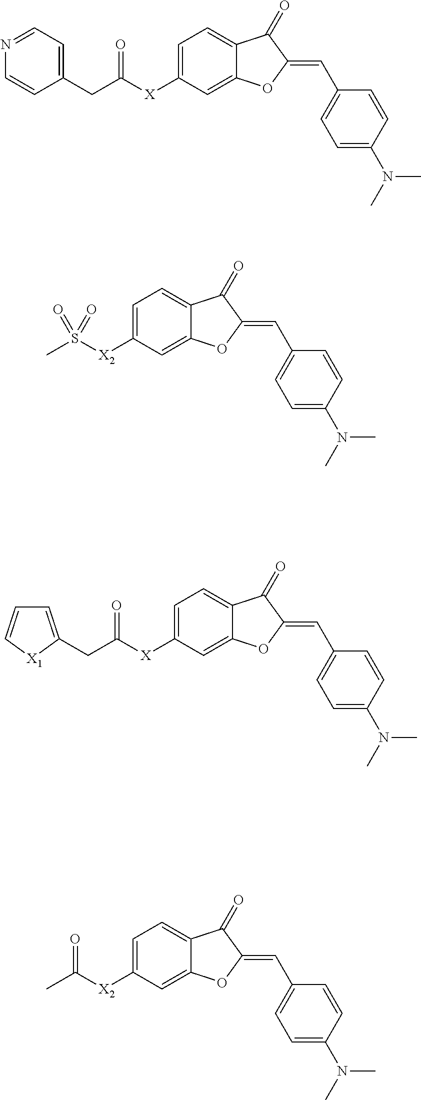
wherein X=O, S, NH, or NMe; X₁=O, S, NH, or NMe; and X₂=O, NH, or NMe.

4. A pharmaceutical composition comprising a compound selected from one of the group consisting of: 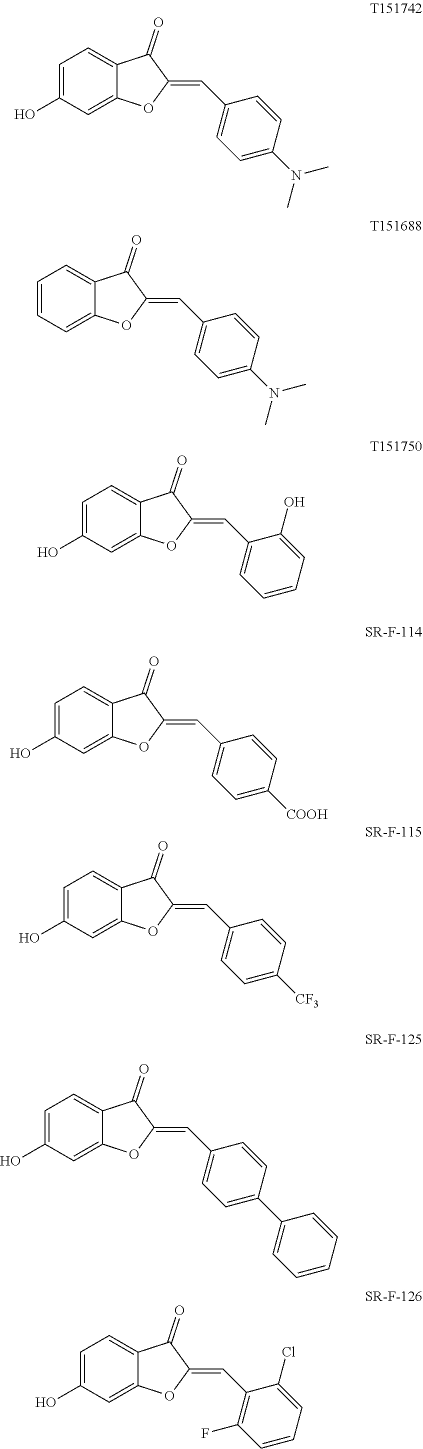 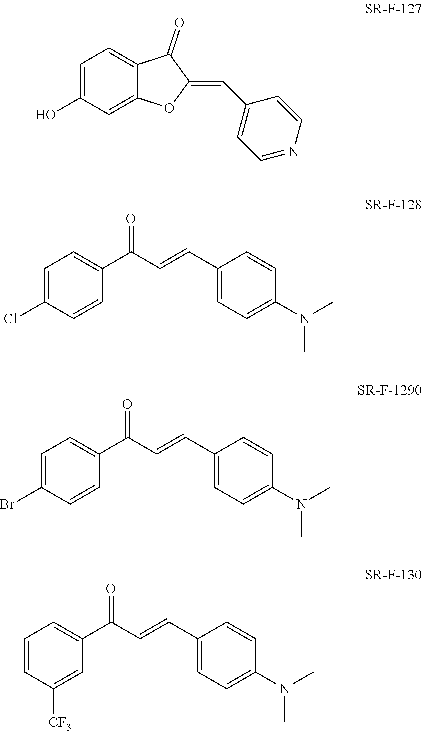 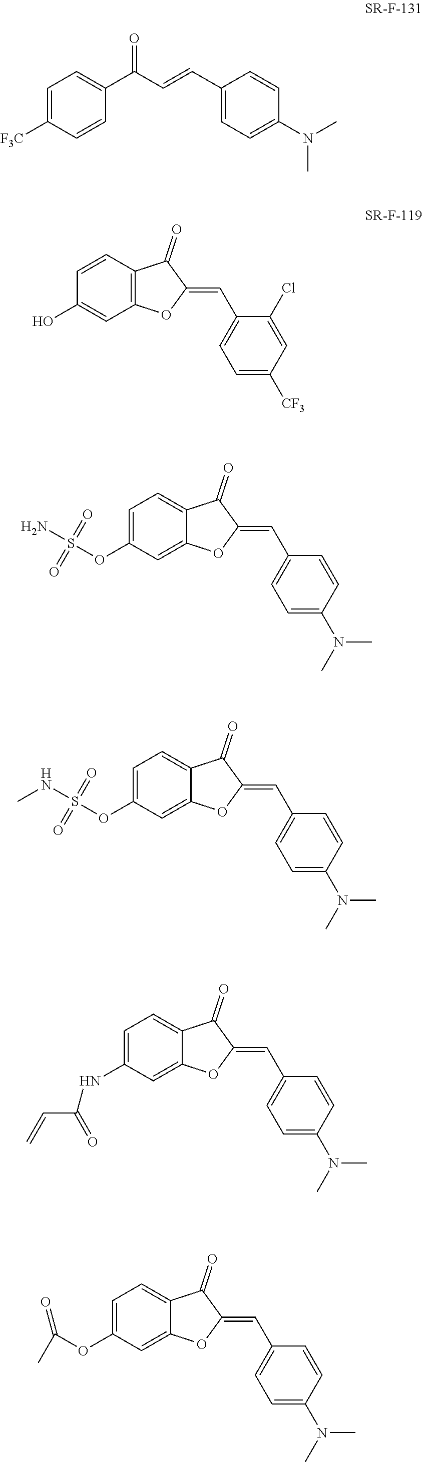 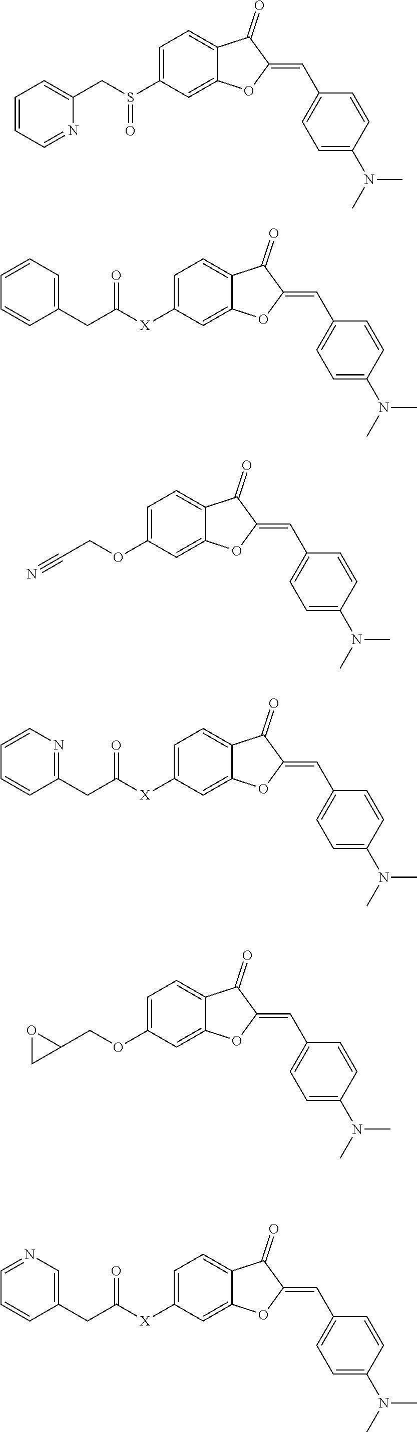 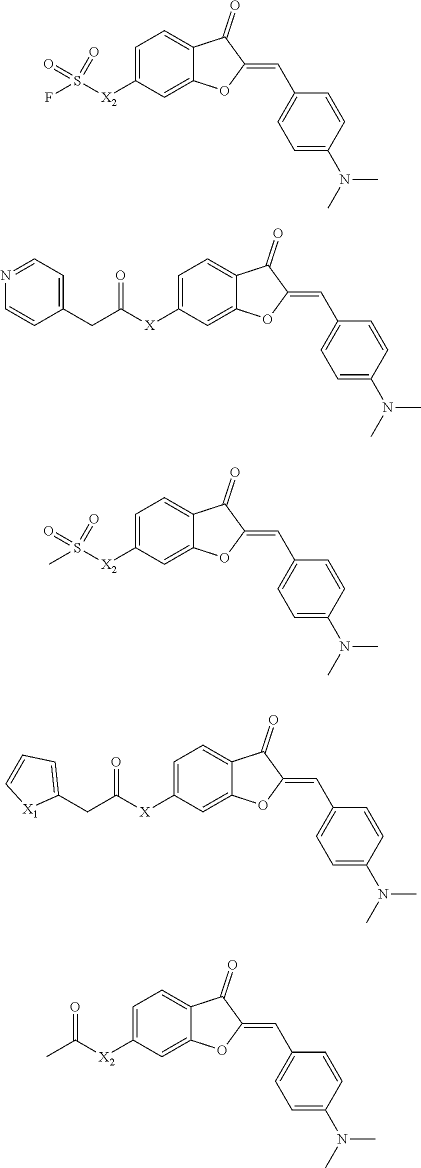
wherein X=O, S, NH, or NMe; X₁=O, S, NH, or NMe; and X₂=O, NH, or NMe.

5. The pharmaceutical composition of claim 4 additionally comprising a pharmaceutically acceptable carrier.

6-7. (canceled)

8. A method of treating a patient having cancer comprising administering to said patient a therapeutically effective amount of the compound of claim 2.

9. A method of treating a patient having cancer comprising administering to said patient a therapeutically effective amount of the pharmaceutical composition of claim 5.

10. A method of treating a patient having cancer comprising administering to said patient a therapeutically effective amount of the compound of claim 3.

11. (canceled)

12. The method of claim 8 wherein said compound targets Ero1α for treatment of said patient having cancer wherein said cancer is lung cancer.

13. The method of claim 9 wherein said compound targets Ero1α for treatment of said patient having cancer wherein said cancer is lung cancer.

14. The method of claim 10 wherein said compound targets Ero1α for treatment of said patient having cancer wherein said cancer is lung cancer.

15. (canceled)

16. A diagnostic method comprising:
determining Ero1-α expression of a tumor cell for use as a diagnostic for administering to a patient an effective amount of an inhibitor compound targeting Ero1 alpha.

17. The diagnostic method of claim 16 including wherein said inhibitor compound is a compound that is selected from one of the group consisting of:

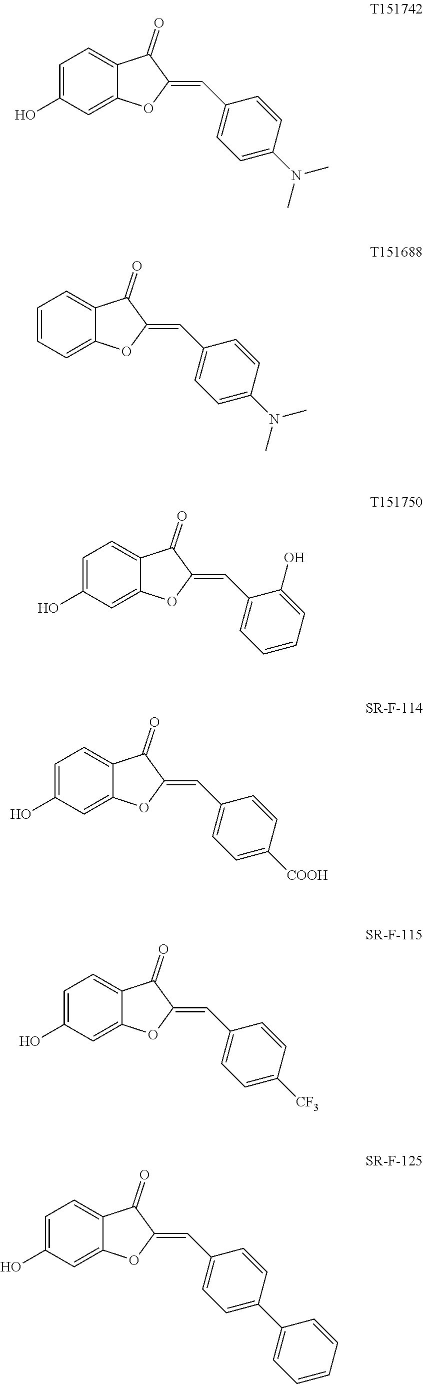 [SMILES: CN(C)c1ccc(/C=C2\Oc3cc(O)ccc3C2=O)cc1 ; CN(C)c1ccc(/C=C2\Oc3ccccc3C2=O)cc1 ; O=C1/C(=C/c2ccccc2O)Oc2cc(O)ccc21 ; O=C(O)c1ccc(/C=C2\Oc3cc(O)ccc3C2=O)cc1 ; O=C1/C(=C/c2ccc(C(F)(F)F)cc2)Oc2cc(O)ccc21 ; O=C1/C(=C/c2ccc(-c3ccccc3)cc2)Oc2cc(O)ccc21]

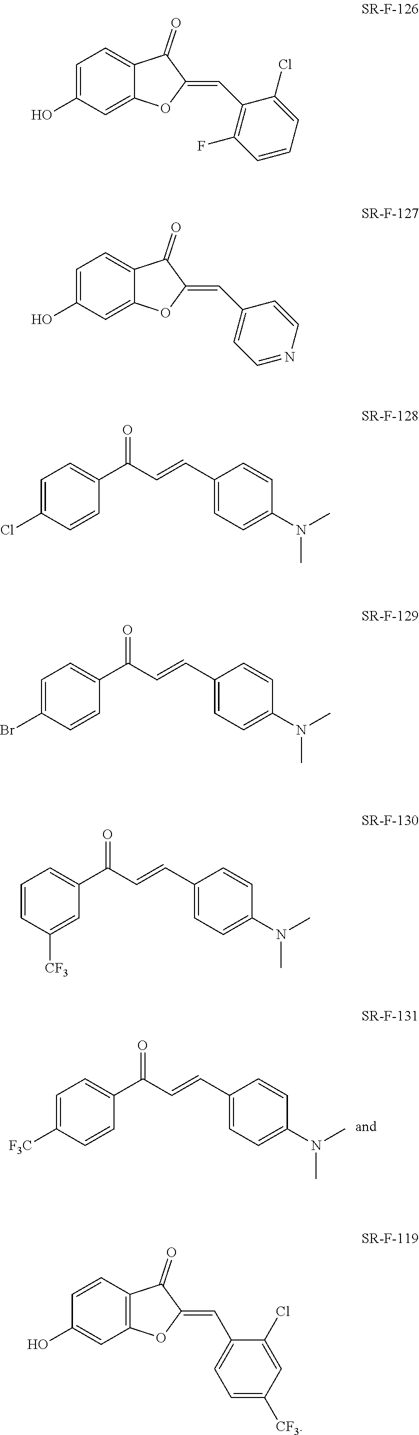

18. The diagnostic method of claim 16 including wherein said inhibitor compound is a compound that is selected from one of the group consisting of: 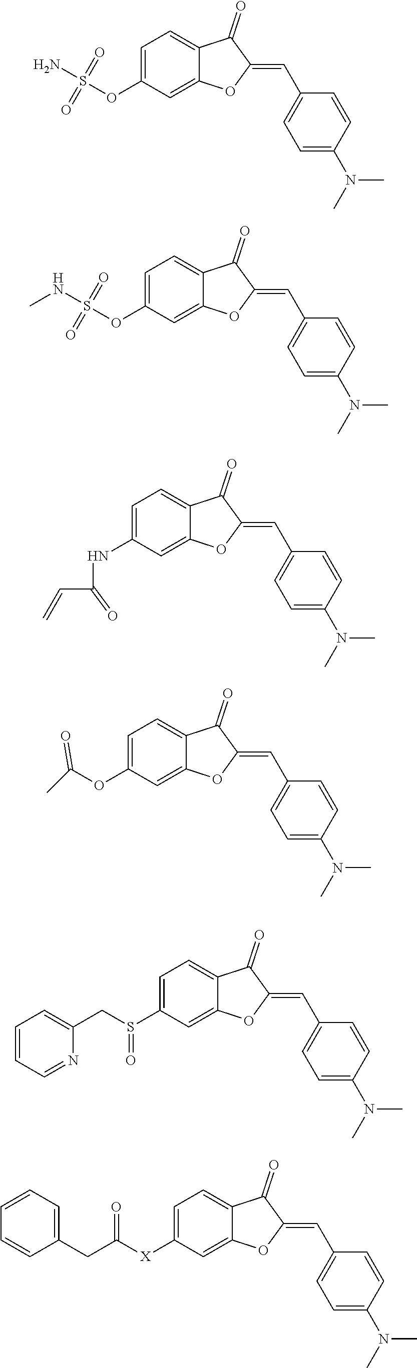 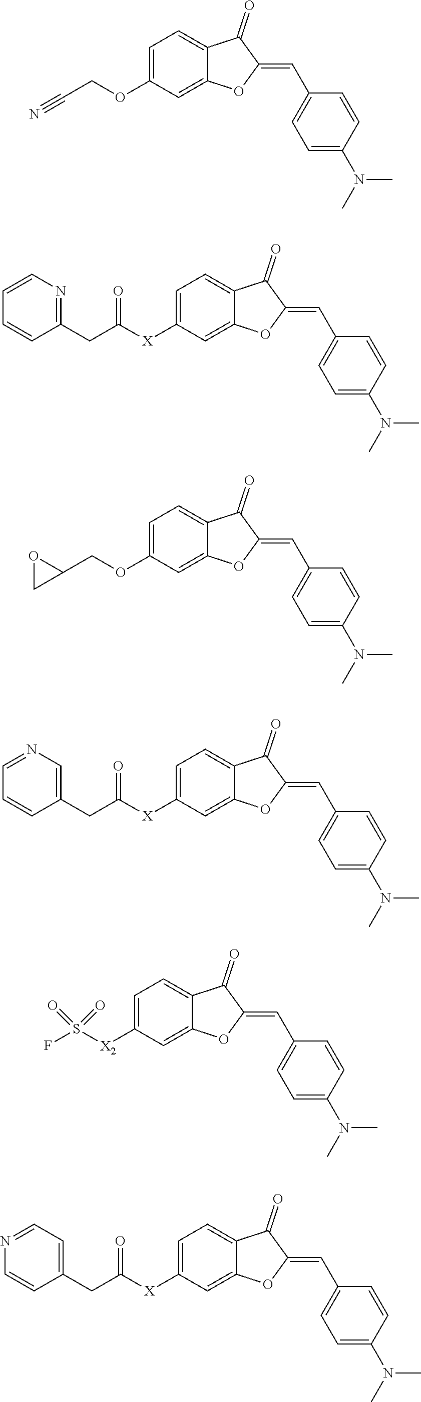 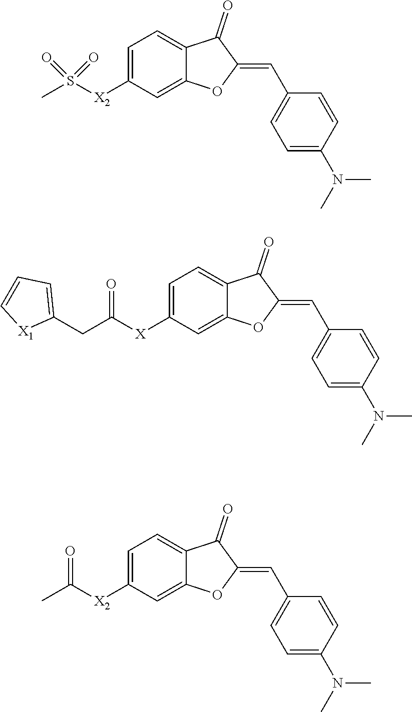
wherein X=O, S, NH, or NMe; X₁=O, S, NH, or NMe; and X₂=O, NH, or NMe.

19. The method of claim 17 wherein said tumor cell is lung cancer.

20. The method of claim 18 wherein said tumor cell is lung cancer.
